(19) United States
(12) Patent Application Publication
(10) Pub. No.: US 2005/0261213 A1
(43) Pub. Date: Nov. 24, 2005

(54) Nucleic acid compositions and methods for use

(76) Inventors: Patrick Branigan, Lansdowne, PA (US); Theresa J. Goletz, King of Prussia, PA (US); David M. Knight, Berwyn, PA (US); Stephen G. McCarthy, West Chester, PA (US); Bernard J. Scallon, Collegeville, PA (US); Linda A. Snyder, Pottstown, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/401,386

(22) Filed: Mar. 28, 2003

Related U.S. Application Data
(63) Continuation-in-part of application No. 10/247,203, now abandoned.
(60) Provisional application No. 60/328,371, filed on Oct. 10, 2001.

(51) Int. Cl. A61K 48/00

(52) U.S. Cl. 514/44

(57) ABSTRACT

Polynucleotide compositions encoding a tumor antigen antigenic determinant and optionally including a nucleic acid adjuvant are disclosed. The compositions are useful for prophylaxis or treatment of cancer.

### CROSS-REFERENCE TO RELATED APPLICATION

This application is a continuation-in-part of U.S. patent application Ser. No. 10/247,203, filed Sep. 19, 2002, which claims the benefit of U.S. Provisional Application No. 60/328,371, filed Oct. 10, 2001.

### FIELD OF THE INVENTION

The present invention relates to nucleic acid vectors comprising sequences that encode a tumor antigen immunogen and their use as cancer treatments. A nucleic acid sequence encoding an adjuvant can optionally be included.

### BACKGROUND OF THE INVENTION

Cancer is a serious disease that afflicts one in four people. In the last fifty years, there have been significant improvements in the early detection of cancer, as well as the development of a number of therapies to treat cancer. Therapies include surgery to remove primary tumors, and sublethal radiation and chemotherapy to treat disseminated disease. While these treatments have resulted in apparent cures for many patients, the treatments can be quite debilitating and are still often ineffective at preventing death from this disease. There is clearly a need for therapies that are less destructive, as well as for novel therapies that harness the body's natural defenses to fight cancer.

Cancer can be divided into two classifications, depending upon the cell type the tumor is derived from. In general, carcinomas are derived from epithelial cells, while sarcomas are derived from mesodermal tissues. Tumor-specific antigens have been identified in both cancer classifications.

Breast cancer is a common malignancy second only to lung cancer among cancer deaths in women. In 2000, it was estimated that 182,800 new cases were diagnosed and 41,200 deaths resulted from breast cancer in the United States (U.S.). Standard-dose combination chemotherapy can yield high response rates in previously untreated patients with metastatic disease, but complete responses are rare. Despite initial chemosensitivity, median disease response duration is less than 1 year due to the emergence of chemoresistant disease. The median survival for patients with metastatic disease has remained approximately 2 years for those treated with standard-dose chemotherapy.

A majority of breast carcinomas express on their surface a protein called mucin 1 (MUC1), a transmembrane protein that is normally expressed in non-disease states on ductal epithelial cells, such as those in the intestinal mucosa exposed to the lumen of the small intestine. The most notable feature of MUC1 is its large extracellular domain, which is comprised of 30-100 tandem repeats of a 20 amino acid sequence. The tandem repeats confer a rigid structure to this portion of the protein, and the repeats are a substrate for heavy glycosylation. In addition, in normal cells MUC1 is only expressed on the ductal side of the cell. It is thought that MUC1 may provide a lubrication function to the duct, and it may also be involved in signal transduction. Because the protein is normally expressed on the ductal side of cells, it is rarely exposed to the outside of the organism, and is considered a “sequestered antigen”, because in its native form MUC1 is not exposed to immune system surveillance.

In contrast, MUC1 expression is different in epithelial tumors. The protein becomes overexpressed and is present all over the surface of the cell, and it is relatively deglycosylated as compared to the normal form expressed in ductal epithelial cells. Thus, the distribution and pattern of expression is very different in normal and neoplastic tissues, and the deglycosylated, aberrant protein exposes novel epitopes to the immune system. Because the pattern of expression is different from normal, it is possible that the immune system can now recognize the tumor-associated MUC1 as foreign and attempt to destroy the cells expressing this protein. Indeed, the immune system does appear to act in this way in some cancer patients. It has been shown that patients with ovarian, breast or pancreatic cancer possess weak antibody and cytotoxic T lymphocyte (CTL) responses to MUC1, indicating that their immune systems do indeed recognize a difference in the tumor-associated MUC1. However, the immune responses are clearly not strong enough to eliminate tumor cells.

These observations have led some investigators to develop therapeutic strategies designed to induce or strengthen the natural immune response by targeting the MUC1 antigen. For example, various immunotherapeutic approaches which target MUC1 have been shown to induce immune responses in mice and chimpanzees (Barratt-Boyes et al., Clinical Cancer Research 5, 1918-1924 (1999); Pecher and Finn, Proc. Natl. Acad. Sci. (USA) 93, 1699-1704 (1996); Gong et al., Nature Medicine 3, 558-561 (1997) data described herein). The immunotherapies tested in the mouse models induced tumor protection. Thus, MUC1 immunotherapeutic strategies are potentially promising approaches for patients with MUC1-expressing tumors who otherwise lack effective treatment options.

Several groups have attempted to use MUC1 peptides to prime a cellular response in patients. This relies on the concept that cells could process the peptide and present it in the context of Class I molecules to the immune system, to cause a Th1 response to cells expressing the MUC1 protein. There are several disadvantages to known approaches. First, peptides have short half-lives, requiring administration of large amounts of the peptide. Second, each person expresses several Class I molecules and a given peptide binds to only one molecule, which will be held by a minority of the patient population. Third, the immunity generated by such approaches may not be relevant to treating such cancers; it has been noted that anti-peptide immunity can be generated by peptide immunization, which does not always lead to anti-protein immunity. In contrast, responses to recombinant vaccine constructs expressing MUC1 have been shown to induce immune responses in mice and chimpanzees. As such, immunotherapeutic strategies targeting the MUC1 antigen are a potentially promising approach for patients with metastatic breast cancer who otherwise lack effective treatment options. This is likewise true for other cancers where MUC1 is overexpressed, such as non-small cell lung cancer, pancreatic cancer, colon cancer, renal cancer and prostate cancer, among others.

Prostate cancer is the second leading cause of cancer-related death in men. Approximately 180,000 men will be diagnosed with prostate cancer each year, and 40,000 succumb to the disease each year. Prostate tumor cells have a low proliferation rate and do not respond to standard chemotherapies, which are most toxic to the most rapidly dividing cells in the body. Instead, prostate cancer can be treated surgically, with radiation therapy or hormonal therapy. Surgery and radiation therapy can lead to undesirable side effects, such as incontinence and impotence. The disease can often be successfully managed with hormonal therapy, which starves the cells for its required growth factors. However, eventually all tumors treated in this way become androgen-independent and there is no effective treatment beyond that point. There is clearly an unmet medical need to treat this disease more effectively.

Prostate tumors and some breast malignancies express prostate specific antigen (PSA), also known as KLK3, on their surface. PSA is a member of a multigene family known as the human kallikrein gene family. There are 15 closely related genes in the family, all of which map to a 300 kb region of human chromosome 19q13.3-q13.4. Kallikreins are secreted serine proteases. All are synthesized as preproenzymes; proenzymes arise after removal of the signal peptide, and the mature active protease arises after removal of a propeptide. The activity of a given kallikrein will be either trypsin-like or chymotrypsin-like, depending upon the nature of the active site.

PSA or KLK3 is a 30 Kd serine protease with chymotrypsin-like activity, which is responsible for cleaving seminogelin I, seminogelin II and fibronectin in seminal fluid. PSA is most highly expressed in the prostate, but it is also expressed at lower levels in breast, salivary gland, and thyroid. Besides prostate cancer, PSA is expressed in some breast malignancies. PSA has become well known as a serum marker for prostate cancer; it is a very important diagnostic for this disease and increasing serum levels of PSA typically correlate well with the severity of the disease. Expression of PSA is not increased in prostate cancer cells versus normal prostate cells; instead as the disease breaches the normal cellular barriers, PSA leaks into the serum. It is unclear if PSA has a role in the etiology of prostate cancer; various reports have indicated that PSA could either enhance or inhibit tumorigenicity. Several CTL epitopes for PSA have been described for the HLA A2 and A3 haplotypes; identification of these epitopes support the possibility of generating therapeutic in vivo CTL by vaccination.

Kallikrein 2 (KLK2), a protein that closely resembles PSA, is also expressed in prostate tumors. KLK2 is the member of the kallikrein family that most closely resembles PSA, with about 80% identity at the amino acid level. Like PSA, KLK2 is expressed highly in the prostate and in prostate cancer, with lower levels of expression in other tissues, such as breast, thyroid, and salivary gland. KLK2 has trypsin-like activity, and one of its activities is to cleave the proenzyme form of PSA to yield the mature enzyme. There is increasing recognition that KLK2 may be a good serum prognostic indicator to monitor the progress of prostate cancer patients, although it is likely to be a supportive diagnostic along with PSA.

The identification of tumor-specific antigens has supported the concept that immunologic strategies could be designed to specifically target tumor cells in cancer patients. Immunologic recognition of tumor antigens has been subsequently documented in many patients with malignancies, particularly in patients with melanoma. However, these responses are muted and are ineffective in eradicating disease. The development of immune tolerance towards malignant cells is due, in part, to the inability of tumor cells to effectively present antigens to the immune system. Therefore, T cells with the capability of recognizing these antigens fail to become activated. A major focus of cancer immunotherapy has been the attempt to introduce tumor antigens into the cancer bearing host such that they may be recognized more effectively and that meaningful antitumor responses can be generated. In this way, native immunity directed against antigens selective for or over-expressed in malignant cells may be amplified and result in tumor rejection. Approaches to induce tumor-specific immunity have included vaccination with tumor cell extracts, irradiated cells, tumor-specific peptides with and without adjuvant, and dendritic cells (DC) pulsed with tumor peptides/proteins, or manipulated to express tumor-specific genes.

Work in animal models and in some clinical trials indicates that DNA immunization may be an effective method to generate immune responses in vivo, particularly for the generation of cellular immune responses against cells expressing the antigen encoded by the vaccine. Vaccination with plasmids encoding antigens results in the expression of the antigen by the inoculated muscle cells. Professional antigen presenting cells, in particular dendritic cells (DC), recruited to the site of injection, also internalize plasmid or encoded antigen, and subsequently present the antigen at sites of T-cell traffic.

Other examples of active immunotherapy include DC therapies, where the patient's professional antigen presenting cells are removed and pulsed with tumor antigen, transfected with tumor RNA/cDNA, or fused with tumor cells. The ex vivo-treated DC are then reinjected into the patient, and are expected to drive a tumor specific immune response. One disadvantage of such approaches is that they amount to designer therapy that would be very costly and require very specialized skills to administer. Such therapies are unlikely in their current form to be widely used.

A third active immunotherapy approach is peptide vaccination. In this approach, tumor-specific peptides or proteins are administered to the patient, with the hope of directly loading antigen-presenting cells in vivo. This approach is more likely to be usable in the clinic than the ex vivo approach described above, but consistent success has not yet been achieved with this strategy. Some problems include the fact that peptides are short-lived in vivo, and therefore require very large doses. In some clinical trials, peptide vaccination engenders anti-peptide immune responses that do not translate into responses against tumors expressing the whole protein from which the peptides were derived.

Accordingly, there is a long-felt and pressing need to discover vaccines and methods that elicit an immune response that is sufficient to treat or prevent various tumor-related human pathologies.

### SUMMARY OF THE INVENTION

One aspect of the invention is a composition comprising a first isolated polynucleotide encoding or complementary to an antigenic determinant of a tumor-associated protein and a second isolated polynucleotide encoding or complementary to a nucleic acid adjuvant.

Another aspect of the invention is a composition comprising a first isolated polynucleotide encoding or complementary to an antigenic determinant of PSA, KLK2 or MUC1; a second isolated polynucleotide encoding or complementary to a nucleic acid adjuvant and at least one promoter.

Another aspect of the invention is a composition comprising a first isolated polynucleotide encoding or complementary to an antigenic determinant of PSA, KLK2 or MUC1; a second isolated polynucleotide encoding or complementary to IL-18 and at least one promoter.

Another aspect of the invention is a composition comprising an isolated polynucleotide encoding or complementary to an antigenic determinant of PSA, KLK2 or MUC1 and a promoter.

### BRIEF DESCRIPTION OF THE FIGURES

FIG. 1 shows tumor incidence in pMUC1/pIL-18 vaccinated female C57B1/6 mice challenged with MUC1⁺ tumor cells.

FIG. 2 shows tumor volume in pMUC1/pIL-18 vaccinated female C57B1/6 mice challenged with MUC1⁺ tumor cells.

FIG. 3 shows tumor incidence in pMUC1/pIL18 vaccinated female C57B1/6 mice rechallenged with MUC1⁺ tumor cells.

FIG. 4 shows tumor incidence in pMUC1/pIL-18 vaccinated C57B1/6 MUC1 transgenic mice challenged with MUC1⁺ tumor cells.

FIG. 5 shows tumor weights in pMUC1/pIL-18 vaccinated C57B1/6 MUC1 transgenic mice challenged with MUC1⁺ tumor cells.

FIG. 6 shows tumor incidence in pMUC1/pIL18 vaccinated C57B1/6 MUC1 transgenic mice rechallenged with MUC1⁺ tumor cells.

FIG. 7 shows tumor incidence in pMUC1/pIL18 vaccinated C57B1/6 MUC1 transgenic mice challenged twice with MUC1⁺ tumor cells and challenged again with MUC1⁻ tumor cells.

FIG. 8 shows tumor incidence in pMUC1/pIL18 vaccinated female C57B1/6 MUC1 transgenic mice challenged with MUC1⁺ tumor cells.

FIG. 9 shows tumor weight in pMUC1/pIL-18 vaccinated female C57B1/6 MUC1 transgenic mice challenged with MUC1⁺ tumor cells.

FIG. 10 shows tumor incidence in pMUC1/pIL18 vaccinated female C57B1/6 MUC1 transgenic mice challenged with MUC1⁺ tumor cells and rechallenged with MUC1⁻ tumor cells.

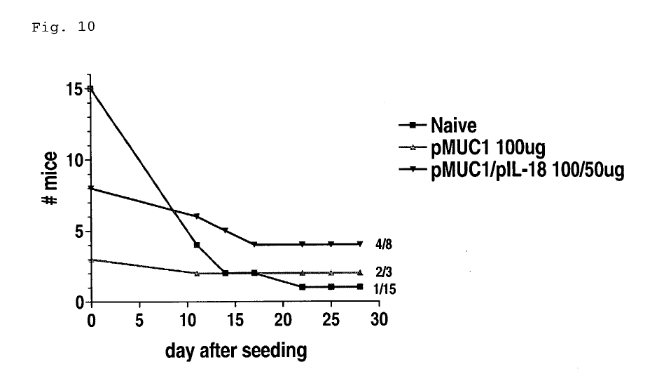

FIG. 11 shows tumor incidence in pMUC1/pIL18 vaccinated male C57B1/6 MUC1 transgenic mice challenged with MUC1⁺ tumor cells.

FIG. 12 shows tumor weights tumor in pMUC1/pIL18 vaccinated male C57B1/6 MUC1 transgenic mice challenged with MUC1+tumor cells.

FIG. 13 shows tumor incidence tumor incidence in pMUC1/pIL18 vaccinated male C57B1/6 MUC1 transgenic mice challenged and rechallenged with MUC1⁺ tumor cells.

### DETAILED DESCRIPTION OF THE INVENTION

All publications, including but not limited to patent and patent applications, cited in this specification are herein incorporated by reference as though fully set forth.

It is further to be understood that all base sizes or amino acid sizes, and all molecular weight or molecular mass values, given for nucleic acids or polypeptides are approximate, and are provided for description.

As used herein, the term “DNA vaccines” or “nucleic acid vaccines” denotes compositions useful for the direct in vivo introduction of DNA encoding an antigen into tissues of a subject for expression of the antigen by tissue cells. DNA vaccines are described in, e.g., International Patent Publication WO 95/20660 and International Patent Publication WO 93/19183.

As used herein, the term “nucleic acid adjuvant” means a nucleotide sequence coding for a protein or protein fragment that enhances an immune response to an antigen.

The present invention provides a composition having a first isolated polynucleotide encoding an antigenic determinant of a tumor protein and a second isolated polynucleotide encoding a nucleic acid adjuvant. The composition also includes promoter polynucleotides that control expression of the first and second polynucleotides.

The invention also provides a composition having an isolated polynucleotide encoding an antigenic determinant of PSA, KLK2 or MUC1 and a promoter controlling expression of the polynucleotide. The composition can further include an isolated polynucleotide encoding an IL-18 adjuvant and a promoter polynucleotide controlling expression of the IL-18. These compositions can be used to elicit an immune response to a cancer-associated tumor protein in a mammal and are useful as nucleic acid vaccines for the treatment and/or prophylaxis of certain cancers or other tumor-related pathologies.

In the present invention, it has been discovered that the compositions of the invention containing a nucleic acid adjuvant can elicit an unexpectedly enhanced immune response in a subject. While not wishing to be bound to any particular mechanism of action, the compositions of the present invention are believed to recruit one or more of B cell, helper T cell, and cytotoxic T cell components of the immune response for effective humoral and cellular immunity when administered to a mammal.

Antigenic determinants useful in the invention are obtained or derived from tumor antigens such as prostate specific antigen (PSA), Kallikrein 2 (KLK2) or mucin-1 (MUC1). These tumor antigens can be of human origin or from closely related species such as Macaca mulatta (Rhesus monkey) or Macaca fascicularis (Cynomologus monkey). The tumor antigens could also be mutated to enhance their immunogenicity. Examples of how the antigen genes could be modified to effect a more robust immune response to the antigen protein include changes that affect antigen gene expression levels, such as addition of intron sequences, alteration or removal of signal sequences required for secretion, or optimization of codons for improved translation. In addition, the antigen gene could be modified to introduce changes to the translated product of the gene, such as alteration or removal of signal sequences required for secretion, addition of ubiquitination signals for degradation, addition of subcellular compartment targeting sequences, addition of molecular chaperone sequences, and optimization of CTL epitope sequences. The antigen genes could be fused together to increase immunogenicity. The CTL/helper epitopes could be linked together, or inserted as part of another molecule, such as an immunoglobulin molecule.

Compositions of the invention including these antigenic determinants are useful for the treatment of any cancer where PSA, KLK2 or MUC1 is uniquely expressed, over-expressed or associated with the presence of tumors caused by the cancer. These cancers include, but are not limited to, prostate, including hormone-refractory prostate cancer (HRPC), and breast cancer.

In an embodiment of the invention, polynucleotides encoding mature PSA of human origin (SEQ ID NOs: 1 and 2), human PSA with introns (SEQ ID NOs: 3 and 4), Macaca mulatta mature PSA (SEQ ID NOs: 5 and 6) or human PSA splice variants (SEQ ID NOs: 7 to 12) are used in the compositions of the invention. Polynucleotides encoding variants of human or Rhesus macaque PSA including one or more of any combination of Thr40, Met112 substitution mutants and/or deletion mutants of one or more of Tyr225, Arg226, Lys227, Trp228, Ile229, Lys230, Asp231, Thr232, Ile233, Val234, Ala235, Asn236 or Pro 237 are also useful in the present invention. Nucleotides encoding at least one antigenic determinant of the molecules disclosed above or the human PSA CTL helper epitopes (SEQ ID NOs: 13 to 22) are also useful in the invention. Further, sequences complementary to any of the polynucleotides disclosed above are also useful in the compositions of the invention.

An exemplary human PSA plasmid construct (SEQ ID NO: 62) encodes the mature form of PSA, an HCMV promoter, Rous Sarcoma Virus enhancer sequence and SV40 polyA site.

In another embodiment of the invention, polynucleotides encoding mature human KLK2 (SEQ ID NOs: 23 and 24), human KLK2 with introns (SEQ ID NO: 25) or human KLK2 splice variants (SEQ ID NOs: 26 to 29) or at least one antigenic determinant thereof are used in the compositions of the invention. Further, sequences complementary to any of the polynucleotides disclosed above are also useful in the compositions of the invention.

In yet another embodiment of the invention, polynucleotides encoding human MUC1 (SEQ ID NOs: 30 and 31), human MUC1 with introns (SEQ ID NO: 32) or human MUC1 splice variants (SEQ ID NOs: 33 to 50) are used in the compositions of the invention. Nucleotides encoding at least one antigenic determinant of these molecules or the human MUC1 CTL helper epitopes (SEQ ID NOs: 51 to 58) or the human MUC1 CD4 T helper epitope (SEQ ID NOs: 59 and 60) are also useful in the compositions of the invention. Further, sequences complementary to any of the polynucleotides disclosed above are also useful in the compositions of the invention.

An exemplary human MUC1 plasmid construct (SEQ ID NO: 61) encodes the mature form of MUC1 and contains an HCMV immediate early (IE) promoter and intron A and an SV40 polyA signal.

Nucleic acid adjuvants useful in the invention are obtained or derived from cytokines such as interleukin-18 (IL-18), interleukin-12 (IL-12) or granulocyte-macrophage colony-stimulating factor (GM-CSF) or costimulatory molecules such as B7-1 (human CD80), a costimulatory ligand for CD28 and CTLA-4 (Thompson, Cell 81:979-982, 1995). These nucleic acid adjuvants can be of human origin or from closely related species such as Macaca mulatta or Cynomologus monkey. The nuleic acid adjuvants could also be mutated to enhance their immunogenicity. Examples of how the antigen genes could be rendered more immunogenic include intron sequences inclusion, alteration or removal of signal sequences required for secretion, optimization of codons for improved translation, addition of ubiquitination signals for degradation, addition of subcellular compartment targeting sequences, addition of molecular chaperone sequences, and optimization of CTL epitopes. The nucleic acid adjuvant genes could be fused together to increase immunogenicity. The CTL/helper epitopes could be linked together, or inserted as part of another molecule, such as an immunoglobulin molecule.

An exemplary cytokine adjuvant is human IL-18 (SEQ ID NOs: 63 and 64). Splice variants of human IL-18 set forth in SEQ ID NOs: 65 to 70 and fragments of human IL-18 encoding at least one antigenic determinant are also useful in the present invention. Rhesus macaque IL-18 (SEQ ID NOs: 71 and 72) is very similar to human IL-18 and can also be used according to the present invention. Further, sequences complementary to any of the polynucleotides disclosed above are also useful in the compositions of the invention.

An exemplary human IL-18 plasmid construct encodes the mature form of IL-18 linked to an immunoglobulin signal sequence. The construct includes a genomic fragment that encodes the 19 residue anti-IL-12 12B75 heavy chain signal sequence (SEQ ID NOs: 73 and 74) linked to a human IL-18 cDNA sequence to ensure production of human IL-18 in any cell type.

Further, IL-18 mutants are useful in the invention. For example, changes in non-surface exposed residues that could be made that would result in the high probability of retention of IL-18 activity with no changes in immunogenicity are Thr¹⁰ for Ser¹⁰); Val¹² for Ile¹²; Ser⁴⁵ for Thr⁴⁵; Tyr⁴⁷ for Phe⁴⁷; Phe⁵² for Tyr⁵²; Val⁶⁴ for Ile⁶⁴; and Tyr¹⁰¹ for Phe¹⁰¹. Changes in amino acids with a low percentage of surface exposure that could be made that would result in the high probability of retention of IL-18 activity with possible changes in immunogenicity are Val⁵ for Leu⁵; Val²⁰ for Leu²⁰; Ile²⁰ for Leu²⁰; Tyr²¹ for Phe²¹; Val²² for Ile²²; Ile⁶⁶ for Val⁶⁶; Thr⁷² for Ser⁷²; and Phe¹⁴⁸ for Ser⁴⁸. Changes that could be made in amino acids involved in receptor contact that would result in alteration of IL-18 activity by either increasing or decreasing binding of the IL-18 analog to the IL-18 receptor are Glu⁴ for Lys⁴; Ile⁶ for Glu⁶; Asp⁸ for Lys⁸; Ile¹³ for Arg¹³; Arg¹⁵ for Leu 15; Lys¹⁷ for Asp¹⁷; Lys²⁷ for Arg²⁷ Ala³⁰ for Phe³⁰; Lys³⁵ for Asp³⁵; Phe³⁷ for Asp³⁷; Glu³⁸ for Cys³⁸; Ala³⁹ for Arg³⁹; Trp⁴⁰ for Asp⁴⁰; Glu⁵¹ for Met⁵¹; Gly⁵³ for Lys⁵³; Ile⁵⁶ for Gln⁵⁶; Ala⁵⁸ for Arg⁵⁸; Lys⁶² for Val⁶²; Lys⁹⁴ for Asp⁹⁴; Phe⁹⁵ for Thr⁹⁵; Leu¹⁰⁴ for Arg¹⁰⁴; Ile¹⁰⁸ for Gly¹⁰⁸; Lys¹¹¹ for Asn¹¹¹; Phe¹²⁹ for Lys 129; Asp¹³¹ for Arg¹³¹; Leu¹³² for Asp¹³²; Glu¹³³ for Leu¹³³; Ala¹³⁴ for Phe¹³⁴; Thr¹⁵⁰ for Met¹⁵⁰; and Ser¹⁵¹ for Phe¹⁵¹.

One exemplary double mutant changes Ile¹¹ for Val¹¹ and Ala⁶³ for Thr⁶³ (IL-18 V11I/T63A) (SEQ ID NOs: 75 and 76). Another exemplary double mutant is Thr¹⁰ for Ser¹⁰ and Ala⁶³ for Thr⁶³ (IL-18 S10T/T63A) (SEQ ID NOs: 77 and 78). An exemplary triple mutant is Thr¹⁰ for Ser¹⁰, Asn¹⁷ for Asp¹⁷ and Ala⁶³ for Thr⁶³ (IL-18 S10T/D17N/T63A) (SEQ ID NOs: 79 and 80). Both double mutants and the triple mutant retain their IL-18 activity.

An exemplary mutant human IL-18 plasmid construct encodes the mature form of an IL-18 mutant linked to an immunoglobulin signal sequence. In an embodiment of the invention, the construct (SEQ ID NO: 81) includes a genomic fragment that encodes the anti-IL-12 12B75 heavy chain signal sequence (SEQ ID NO: 73) linked to the IL-18 V11I/T63A double mutant cDNA sequence (SEQ ID NO: 75) to ensure production of the mutant human IL-18 in any cell type. The plasmid also contains an E. coli ori, an HCMV IE promoter and minimal rabbit β-globin (mRBG) polyA.

In a specific embodiment of the invention, a composition comprising a first polynucleotide encoding a human MUC1 having a nucleotide sequence as set forth in SEQ ID NO: 30 and a second polynucleotide encoding the IL-18 V11I/T63A double mutant having a nucleotide sequence as set forth in SEQ ID NO: 75 is prepared. For example, a composition comprising the polynucleotide having the sequence set forth in SEQ ID NO: 61 and the polynucleotide having the sequence set forth in SEQ ID NO: 81 is prepared.

In another specific embodiment of the invention, a composition comprising a first polynucleotide encoding a human PSA having a nucleotide sequence as set forth in SEQ ID NO: 1 and a second polynucleotide encoding the IL-18 V11I/T63A double mutant having a nucleotide sequence as set forth in SEQ ID NO: 75 is prepared. For example, a composition comprising the plasmid having the polynucleotide sequence set forth in SEQ ID NO: 62 and the plasmid having the polynucleotide sequence set forth in SEQ ID NO: 81 is prepared.

In the compositions of the invention, the first and second polynucleotides can be contained on the same nucleic acid vector or can be contained on separate nucleic acid vectors.

The tumor antigen encoding nucleic acid used in the invention may be isolated from patients having a tumor-related cancer, preferably from the cancerous tissue itself or from mRNA or cDNA encoding a cancer-related tumor protein or antigenic portion thereof. Alternatively, the different tumor antigen nucleic acids can be obtained from any source and selected based on screening of the sequences for differences in coding sequence or by evaluating differences in elicited humoral and/or cellular immune responses to multiple tumor sequences, in vitro or in vivo, according to known methods.

The present invention can also include polynucleotides encoding polypeptides having immunogenic activity elicited by an amino acid sequence of a tumor amino acid sequence as at least one epitope or antigenic determinant. Such amino acid sequences substantially correspond to at least one 10-200 amino acid fragment and/or consensus sequence of a known tumor antigen protein sequence, as described herein or as known in the art. Such a tumor antigen sequence can have overall homology or identity of at least 50% to a known tumor protein amino acid sequence, such as 50-99% homology, or any range or value therein, while eliciting an immunogenic response against at least one type of tumor protein, preferably including at least one pathologic form.

Percent homology can be determined, for example, by comparing sequence information using the GAP computer program, version 6.0 available from the University of Wisconsin Genetics Computer Group (UWGCG). The GAP program utilizes the alignment method of Needleman and Wunsch (J. Mol. Biol. 48, 443 (1970)), as revised by Smith and Waterman (Adv. Appl. Math. 2, 482 (1981)). Briefly, the GAP program defines similarity as the number of aligned symbols (i.e., nucleotides or amino acids) which are similar, divided by the total number of symbols in the shorter of the two sequences. The preferred default parameters for the GAP program include: (1) a unitary comparison matrix (containing a value of 1 for identities and 0 for non-identities) and the weighted comparison matrix of Gribskov and Burgess, Nucl. Acids Res. 14, 6745 (1986), as described by Schwartz and Dayhoff, eds., Atlas of Protein Sequence and Structure, National Biomedical Research Foundation, Washington, D.C. (1979), pp. 353-358; (2) a penalty of 3.0 for each gap and an additional 0.10 penalty for each symbol in each gap; and (3) no penalty for end gaps.

In another embodiment, a vector of the present invention comprises a pathologic form of at least one tumor protein. Examples of such sequences are readily available from commercial and institutional tumor sequence databases, such as GENBANK, or other publically available databases. Nucleic acid substitutions or insertions to either a tumor protein sequence or a cytokine sequence to modify the tumor or cytokine to obtain an additional tumor or cytokine protein, encoded by a nucleic acid for use in a viral or nucleic acid vaccine of the present invention, can include substitutions or insertions of at least one amino acid residue (e.g., 1-25 amino acids). Alternatively, at least one amino acid (e.g., 1-25 amino acids) can be deleted from a tumor or cytokine sequence. Preferably, such substitutions, insertions or deletions are identified based on the sequence determination of proteins obtained by nucleotide sequencing of at least one tumor protein or immune adjuvant encoding nucleic acid from an individual.

Non-limiting examples of such substitutions, insertions or deletions preferably are made by the amplification of DNA or RNA sequences from a tumor or other cell line, which can be determined by routine experimentation to provide modified structural and functional properties of a tumor protein or protein adjuvant. Variants can also be made by making mutations in the coding sequence of the tumor protein or protein adjuvant. The tumor protein or protein adjuvant sequences so obtained preferably have different antigenic or adjuvant properties from the original tumor protein or adjuvant protein. Such antigenic differences can be determined by suitable assays, e.g., by determining differences in antigen-specific induced proliferation or cytotoxicity assays, or by differences in antigen-specifc induced humoral response, following vaccination with the modified tumor protein or adjuvant protein nucleic acid sequences encoded by vectors described in the present invention.

Any substitution, insertion or deletion can be used as long as the resulting tumor and adjuvant proteins or antigenic determinants thereof, encoded by nucleic acids, elicits an immune response which targets the tumor cells expressing the tumor antigen. Each of the above substitutions, insertions or deletions can also include modified or unusual amino acids as are known to those skilled in the art.

The attached Sequence Listing presents non-limiting examples of alternative nucleic acid sequences (recited as DNA sequences, but also including the corresponding RNA sequence (where U is substituted for T in the corresponding RNA sequence)) of tumor antigen proteins, as well as adjuvant nucleic acid sequences, that can be encoded by a polynucleotide according to the present invention. Such compositions can comprise at least one tumor antigen protein encoding nucleic acid and at least one adjuvant protein encoding nucleic acid, and can include linear or circular DNA or RNA, optionally further comprising additional regulatory sequences, such as but not limited to promoters, enhancers, selection, restriction sites, and the like, as is well known in the art. For amino acid sequences, any suitable codon can be used for expression, preferably human preferred codons as is well known in the art (see, e.g., the Appendices in Ausubel et al., eds., Current Protocols in Molecular Biology, Greene Publishing Co., New York, (1987-1995)) and such sequences can be further modified, e.g., where specific antigenic sequences can be used.

Accordingly, based on the disclosed non-limiting examples of specific substitutions, alternative substitutions can be made by routine experimentation, to provide alternative tumor/adjuvant vaccines of the present invention, e.g., by making one or more substitutions, insertions or deletions in proteins or tumor proteins which give rise to effective immune responses.

As is well known in the art, a large number of factors can influence the efficiency of expression of antigen genes and/or the immunogenicity of DNA vaccines. Examples of such factors include the reproducibility of inoculation, construction of the plasmid vector, choice of the promoter used to drive antigen gene expression and stability of the inserted gene in the plasmid. Depending on their origin, promoters differ in tissue specificity and efficiency in initiating mRNA synthesis (Xiang et al., Virology, 209, 564-579 (1994); Chapman et al., Nucl. Acids. Res., 19, 3979-3986 (1991)). To date, most DNA vaccines in mammalian systems have relied upon viral promoters derived from strains of cytomegalovirus (CMV). These have had good efficiency in both muscle and skin inoculation in a number of mammalian species.

Another factor known to affect the immune response elicited by DNA immunization is the method of DNA delivery; parenteral routes can yield low rates of gene transfer and produce considerable variability of gene expression. High-velocity inoculation of plasmids, using a gene-gun, enhanced the immune responses of mice (Eisenbraun et al., DNA Cell Biol., 12, 791-797 (1993)), presumably because of a greater efficiency of DNA transfection and more effective antigen presentation by dendritic cells. Vectors containing the nucleic acid-based vaccine of the invention may also be introduced into the desired host by other methods known in the art, e.g., transfection, electroporation, microinjection, transduction, cell fusion, DEAE dextran, calcium phosphate precipitation, lipofection, liposome fusion, transdermal patch, or a DNA vector transporter (see, e.g., Wu et al., J. Biol. Chem. 267, 963-967 (1992); Wu and Wu, J. Biol. Chem. 263, 14621-14624 (1988); Hartmut et al., Canadian Patent Application No. 2,012,311, filed Mar. 15, 1990), or any other known method or device.

The compositions of the invention could be contained within one or more cellular delivery vectors such as plasmids, mammalian viruses, bacteria or mammalian cells having appropriate regulatory and control elements as are well known to those skilled in the art. For example, expression of the tumor antigen and nucleic acid adjuvant polynucleotide sequences could be under the control of a suitable promoter such as the human cytomegalovirus immediate early (HCMV IE) promoter or dihydrofolatereductase promoter and a polyadenylation (polyA) signal such as the SV40 late, SV40 early polyA signal or a synthetic polyA sequence. An intron may be included for enhanced expression, such as the HCMV TE intron A or natural introns from the antigen or adjuvant genes.

An exemplary plasmid useful with the compositions of the invention contains an E. coli origin of replication, an aph(3′)-la kanamycin resistance gene, HCMV immediate early promoter with intron A, a synthetic polyA sequence and a bovine growth hormone terminator. Another exemplary plasmid contains an E. coli origin of replication, an ant(4′)-la kanamycin resistance gene, Rous sarcoma virus long terminal repeat sequences, HCMV immediate early promoter and an SV40 late polyA sequence.

Examples of suitable viruses that can act as recombinant viral hosts for the compositions of the invention include vaccinia, canarypox, and adenovirus, as are known in the art. Various genetically engineered virus hosts (“recombinant viruses”) can also be used. Viral cellular delivery vectors containing the compositions of the invention can promote a suitable immune response that targets activation of B lymphocytes, helper T lymphocytes, and cytotoxic T lymphocytes.

A preferred recombinant virus for use with the compositions of the invention is vaccinia virus (International Patent Publication WO 87/06262; Cooney et al., Proc. Natl. Acad. Sci. USA 90, 1882-1886 (1993); Graham et al., J. Infect. Dis. 166, 244-252 (1992); McElrath et al., J. Infect. Dis. 169, 41-47 (1994)). In another embodiment, recombinant canarypox can be used (Pialoux et al., AIDS Res. Hum. Retroviruses 11, 373-381 (1995), erratum in AIDS Res. Hum. Retroviruses 11, 875 (1995); Andersson et al., J. Infect. Dis. 174, 977-985 (1996); Fries et al., Vaccine 14, 428-434 (1996); Gonczol et al., Vaccine 13, 1080-1085 (1995)). Another alternative is defective adenovirus or adenovirus (Gilardi-Hebenstreit et al., J. Gen. Virol. 71, 2425-2431 (1990); Prevec et al., J. Infect. Dis. 161, 27-30 (1990); Lubeck et al., Proc. Natl. Acad. Sci. USA 86, 6763-6767 (1989); Xiang et al., Virology 219, 220-227 (1996)). Other suitable viral vectors include attenuated or defective DNA virus, such as but not limited to herpes simplex virus (HSV) (see, e.g., Kaplitt et al., Molec. Cell. Neurosci. 2, 320-330 (1991)), papillomavirus, Epstein Barr virus (EBV), see, e.g., U.S. Pat. Nos. 5,990,091; 5,766,599; 5,756,103; 6,086,890; 6,274,147; 5,585,254; 6,140,114; 5,616,326; 6,099,847; 6,221,136; 6,086,891; 5,958,425; 5,744,143; 5,558,860; 5,266,489; 5,858,368; 5,795,872; 5,693,530; 6,020,172 and the like.

Another aspect of the present invention concerns engineering of bi-functional plasmids that can serve as a composition of the invention and a recombinant virus vector. Direct injection of the purified plasmid DNA, i.e., as a DNA vaccine, would elicit an immune response to the antigen expressed by the plasmid in test subjects. The plasmid would also be useful in live, recombinant viruses as immunization vehicles.

The bi-functional plasmid of the invention provides a heterologous gene, or an insertion site for a heterologous gene, under control of two different expression control sequences: an animal expression control sequence, and a viral expression control sequence. The term “under control” is used in its ordinary sense, i.e., operably or operatively associated with, in the sense that the expression control sequence, such as a promoter, provides for expression of a heterologous gene. In another embodiment, the animal expression control sequence is a mammalian promoter (avian promoters are also contemplated by the present invention); in a specific embodiment, the promoter is a late or early SV40 promoter, cytomegalovirus immediate early (CMV) promoter, a vaccinia virus early promoter, or a vaccinia virus late promoter, or any combination thereof. Subjects could be vaccinated with a multi-tiered regimen, with the bi-functional plasmid administered as DNA and, at a different time, but in any order, as a recombinant virus vaccine. The invention contemplates single or multiple administrations of the bi-functional plasmid as a DNA vaccine or as a recombinant virus vaccine, or both. This vaccination regimen may be complemented with administration of viral vaccines or may be used with additional vaccine vehicles containing, e.g., an adjuvant molecule.

As one of ordinary skill in the art can readily appreciate, the bi-functional plasmids of the invention can be used as nucleic acid vaccine vectors. Thus, by inserting at least 1 to about 50 different tumor genes into bi-functional plasmids, a corresponding set of bi-functional plasmids useful as a composition of the invention can be prepared.

The compositions of the invention can be formulated in a pharmaceutically acceptable carrier or diluent. For example, plasmids containing the compositions of the invention could be formulated in microparticles, or with lipid, buffer or other excipients or chemical adjuvants that could aid delivery of DNA, maintain its integrity in vivo, or enhance the immunogenicity of the vaccine. Chemical adjuvants can include compounds or mixtures that enhances the immune response to an antigen. A chemical adjuvant can serve as a tissue depot that slowly releases the antigen and also as a lymphoid system activator that non-specifically enhances the immune response (Hood et al., Immunology, Second ed., (1984), Benjamin/Cummings: Menlo Park, Calif., p. 384). Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, and useful human adjuvants such as BCG (bacille Calmette-Guerin) and Corynebacterium parvum. Selection of an adjuvant depends on the subject to be vaccinated. Preferably, a pharmaceutically acceptable adjuvant is used. For example, a vaccine for a human should avoid oil or hydrocarbon emulsion adjuvants, including complete and incomplete Freund's adjuvant. One example of an adjuvant suitable for use with humans is alum (alumina gel). In a specific embodiment, compositions of the invention are administered intramuscularly in alum. Alternatively, the compositions of the invention can be administered subcutaneously, intradermally, intraperitoneally, intramuscularly or via other acceptable vaccine administration routes.

In the method of the invention, the compositions of the invention can be temporally administered in different orders, or administered in different places in the body at the same time. The compositions of the invention could also be delivered by direct injection into muscle, skin, lymph node, or by application to mucosal surfaces. In a specific embodiment, compostions of the invention are provided intramuscularly (i.m.). Other potential modes of delivery would include injection of DNA, followed by electroporation to enhance cellular uptake and expression of DNA. For screening anti-tumor activity of sera or cells from an individual immunized with a vaccine of the invention, any suitable screening assay can be used, as is known in the art.

Each dose of the composition of the invention may contain at least 1 to about 50 nucleic acid sequences encoding the same or different tumor antigens or portions thereof. Alternatively, the tumor sequences in subsequent compositions may express different tumor genes or portions thereof. In yet another embodiment, the subsequent compositions may have some tumor sequences in common, and others that are different, from the earlier composition. For example, the priming composition may contain nucleic acids expressing tumor proteins arbitrarily designated 1-2. A second (booster) composition may contain polynucleotides encoding tumor proteins 3-5 or 6-10, etc.

In a further embodiment, the prophylactic or therapeutic method of eliciting an immune response to a tumor comprises administering an effective amount of another (e.g., second) composition comprising at least 1 to about 100 different tumor protein fragments or variants, in which the fragments or variants relate to different tumor nucleic acid or amino acid sequences, preferably related to a cancer-associated or pathology-associated tumor protein or antigen sequence.

Any of the vaccine strategies provided herein or known in the art can be provided in any order. The terms “priming” or “primary” and “boost” or “boosting” are used herein to refer to the initial and subsequent immunizations, respectively, i.e., in accordance with the definitions these terms normally have in immunology. For example, a subject may be primed with a composition of the invention, followed by boosting with a composition of the invention or a protein vaccine. Preferably, the composition of the invention is administered intramuscularly. Preferably, the composition of the invention is in the form of a plasmid and is administered with a gene gun or injector pen, needled or needleless. However, other forms and administration are also suitable and included in the present invention.

As can be appreciated by the skilled artisan, the immunization methods of the present invention are enhanced by use of primer, booster or additional administrations of a composition of the present invention. The composition of the invention can be used as a boost, e.g., as described above with respect to the tumor proteins. Alternatively, the composition of the invention can be used to prime immunity, with subsequent administrations used to boost the anti-tumor immune response. The composition of the invention may comprise one or more vectors for expression of one or more tumor proteins or portions thereof. In a preferred embodiment, vectors are prepared for expression as part of the composition of the invention.

The vectors used in the invention could be encoded by plasmids, viruses, bacteria or mammalian cells. The vaccination regimen could be comprised of any or all of these agents, such as a plasmid DNA priming vaccination, followed by a viral vector boost. The latter approach appears to be effective in generating cellular responses important in controlling infectious diseases, and may be very useful in anti-cancer applications of this technology as well.

The present invention confers certain advantages. A first advantage is that administration of the compositions of the invention to an animal results in epitope spreading. This phenomenon is well documented in animal autoimmune disease models (Lehmann et al., Nature 358, 155-157 (1992) and Vanderlugt et al., Curr. Opin. Immunol. 8, 831-836 (1996)). In these models, animals are first immunized with a self-protein or peptide against which they develop immunity, and the immune response causes the destruction of normal tissue expressing the native protein. After tissue destruction, the immune response broadens to include antigens that the animals were not immunized against but which are expressed by the target tissue. If such a process could be duplicated in humans, DNA vaccination could be very effective at inducing immunity to MUC1, KLK2 or PSA as well as to other unique determinants expressed by tumor cells, and broadening the immune response should only be helpful to patient therapy. In addition, tumor cells are continuously changing in response to environmental pressures, and therapy against one antigen could lead to remission until escape variants arise that no longer express that antigen. With epitope spreading, the immune response broadens to include other antigens and theoretically should improve the chances that the tumor cells will be unable to escape the vigilance of the immune system.

Another advantage of the present invention includes the use of a human IL-18 construct that encodes the mature form of IL-18 linked to an immunoglobulin signal sequence. IL-18 is ordinarily expressed as a precursor protein that is not functional until it is cleaved into its mature form by caspase (Gu et al., Science 275, 206-209 (1997) and Ghayur et al., Nature 386, 619-623 (1997)). Most cells do not express caspase, therefore one strategy to ensure IL-18 expression in any cell type is to engineer the protein so that it does not require caspase cleavage for maturation. This strategy was effective for both the human and mouse IL-18 genes.

Another advantage of the present invention is the ability to encode more than one gene on a plasmid or DNA vehicle to enable delivery of more than one protein product to a target tissue/cell Cohen et al., FASEB J. 12, 1611-1626 (1998)). This should ensure that a target tissue expresses all desired proteins with the expectation of a more efficient induction of immune response. For example, we have constructed a double cistron vector, and for example we have shown that it is capable of expressing mouse or human IL-12. IL-12 is a protein comprised of two subunits that must be co-expressed in the same cell in order for the mature molecule to be produced. The two protein subunits are encoded by different genes, and we have shown in tissue culture that a double cistron vector encoding both genes results in more effective production of the mature protein than using two plasmids which encode either gene alone (Cohen, supra).

Another advantage is that a DNA vaccination approach could be very effective in treating cancer patients. In this treatment, the vaccine would be comprised of plasmids (or other DNA-containing agents) that encode antigen(s) specific to prostate or other cancers. The plasmids would be injected into the patient, and the cancer-specific antigens would then be expressed and presented to the immune system. The antigen-presentation process would engender a specific cellular and/or humoral response that could help to control the growth of the tumor or its metastases. From preclinical models there is reason to believe that such an approach could be effective. For example, vaccination of rhesus monkeys with DNA vaccines encoding PSA+/− cytokine adjuvants drives PSA-specific humoral responses and cellular proliferation. In two male monkeys vaccinated in this way, there was evidence of infiltrating cells within the prostate post vaccination, but not in a nonvaccinated control. In experimental work presented herein, it has been shown that vaccination with DNA encoding a different tumor associated antigen, MUC1, can lead to immune responses protective against tumor challenge with MUC1-expressing tumors. Thus, it may be possible to use DNA vaccines to break tolerance to self-antigens that happen to be strongly expressed by tumors, and mount a therapeutic immune response.

While vaccination with a single antigen such as PSA with or without adjuvants may very well be effective as an immunotherapy, it is possible that this would not be enough to control tumor growth. It is entirely possible that an effective immune response against PSA would eliminate PSA+ tumor cells but leave PSA− prostate tumor cells intact and able to grow unfettered. Therefore, it may be desirable to vaccinate with more than one tumor antigen. We propose that a DNA vaccine comprised of cancer antigen with other antigens expressed highly in the cancer, such as KLK2 and/or MUC1 for prostate cancer, and perhaps with other adjuvant/costimulatory genes, would be a more effective approach than vaccination with a single antigen.

Another advantage is that treatment with the compositions of the present invention offers the possibility that cancer patients could develop long-lasting and vigorous immune responses against their tumors that would prolong life, slow disease progression, and possibly eradicate disease. When used as an adjunct therapy, treatment with the compositions of the invention may increase quality of life by minimizing the toxicity of other conventional therapies.

The present invention will now be described with reference to the following specific, non-limiting example.

### EXAMPLE

Generation of Protective Anti-Tumor Immune Response in Mice

Nine groups of female C57B1/6 mice (B6 mice) were vaccinated three times (day −28, −14 and −7) with either vehicle control, empty vector, MUC1-containing vector or IL-18-containing vector, singly or in combination. The MUC1-containing vector (pMUC1) elements were human MUC1, kanamycin resistance gene, Rous Sarcoma virus enhancer, HCMV promoter and SV40 polyA site. The IL-18-containing vector (pIL-18) elements were mouse IL-18, kanamycin resistance gene, Rous Sarcoma virus enhancer, HCMV promoter and SV40 polyA site.

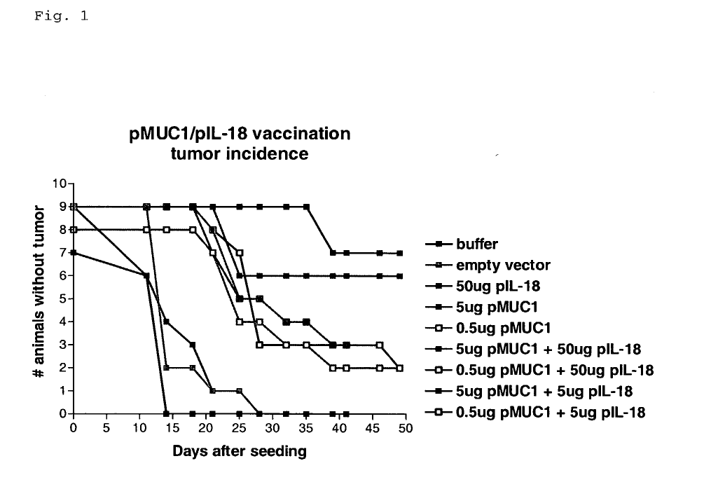

The mice were challenged with syngeneic MUC1⁺ tumor cells (Robbins et al., Cancer Res. 51, 3657-3662 (1991) and Akagi et al., J. Immunother. 20, 38-47 (1997)) by subcutaneous injection on Day 0 and were monitored for tumor incidence and tumor volume for 50 days thereafter. The results in FIG. 1 show that none of the mice in the groups receiving vehicle, empty plasmid or pIL-18 were protected from developing tumors. Two groups received suboptimal doses of pMUC1 and only 2-3 mice were protected. Of the groups vaccinated with the various combinations of pMUC1 and pIL-18 plasmids, those groups receiving the higher dose of pMUC1 in combination with either dose of pIL-18 showed good protection (6 of 9 or 7 of 9 mice). These results are significantly different from the control results (p=0.011 or p=0.003).

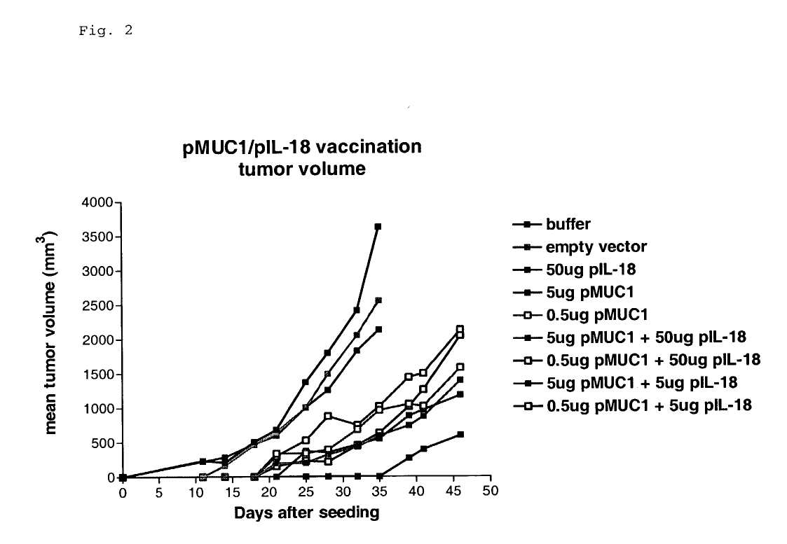

Tumor volume was also evaluated and is shown in FIG. 2. The best result was seen in the group receiving 5 ug pMUC1/5 ug pIL-18, where tumor growth appeared to be delayed to day 35. At that time the slope of tumor growth parallels that of the other groups.

Sera from the animals was collected pre-study, and at days 13, 26 and 34 during and after vaccination. Sera were tested for the presence of anti-MUC1 antibodies, but only low titers were seen. This result indicates that a strong anti-MUC1 antibody response was not responsible for the protection seen in these animals.

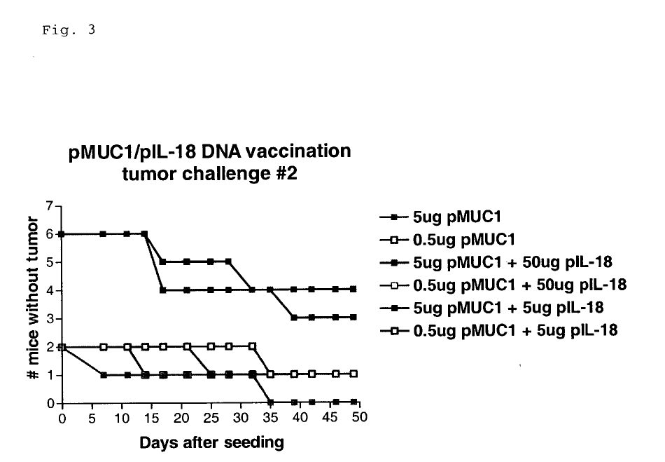

In order to determine if the surviving mice had developed a protective anti-tumor immune response that could be recalled, the mice free of tumors were subjected to a second challenge with MUC1⁺ tumor cells on Day 49 (denoted Day 0 in the results shown in FIG. 3). Mice were monitored an additional 49 days after the second challenge. The results indicate that the group that originally received 5 ug of each test plasmid fared well, with 4 of the original 9 mice protected for another 49 days, while in the group receiving 5 ug pMUC1 and 50 ug pIL-18, 3 of the original 9 mice were still protected. This result indicates that some of the rechallenged mice had developed a protective cellular immune response, since they were able to fend off a second challenge of tumor cells.

The above study showed that while neither plasmid alone offered much protection from tumor challenge, and thus did not prime the immune response particularly well, vaccination with both plasmids at certain doses could indeed lead to protection from tumor challenge, or at least a delay in tumor development indicating that MUC1 and IL-18 plasmids synergize to induce the formation of a protective anti-tumor immune response.

In order to determine if the results were reproducible in a model system more reflective of the human patient, a strain of B6 mice transgenic for human MUC1 (MUC1 Tg mice) (Peat et al., Cancer Res. 52, 1954-1960 (1992); Rowse et al., Cancer Res. 58, 315-321 (1998); and Tempero et al, Int. J. Cancer 80, 595-599 (1999) was used. This model would also allow determination of whether tolerance to a self-antigen could be broken. Doses of pMUC1 were increased in the study while pIL-18 was tested at the same doses. MUC1 Tg mice were vaccinated three times at Day −28, −14 and −7. The mice were challenged with MUC1⁺ tumor cells on Day 0 and monitored for tumor incidence for 28 days.

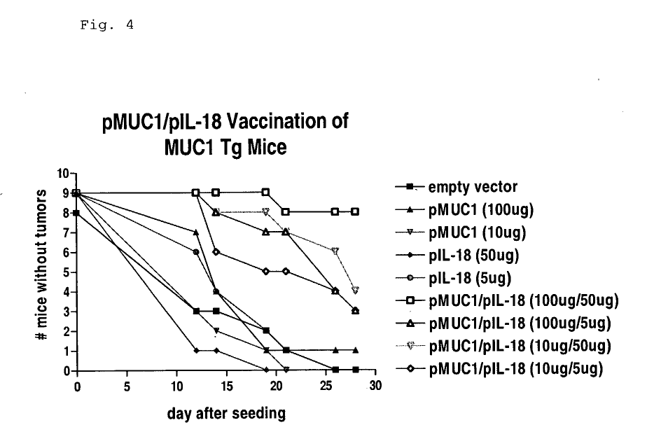

The results in the second study were consistent with the first and are shown in FIG. 4. Animals receiving empty plasmid showed no protection from tumor challenge. Only one animal receiving the higher dose of pMUC1 was protected, while none of those receiving pIL-18 alone were protected. In contrast, the groups receiving the combinations of pMUC1/pIL-18 showed notable protection, particularly the group receiving the highest dose of each plasmid (8/9 without tumors; p=0.002).

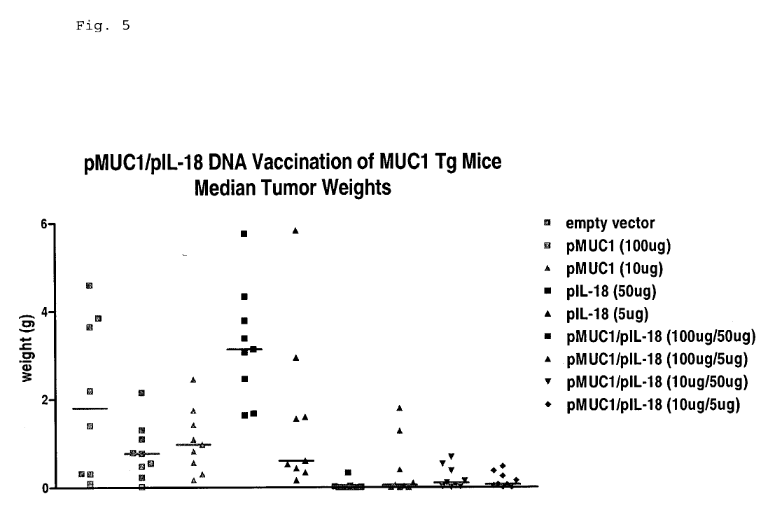

On day 28 the tumors were excised and weighed, as shown in FIG. 5 (horizontal bars are median values). Neither the pMUC1 nor pIL-18 groups had mean weights that were significantly different from the empty vector control group. However, all four pMUC1/pIL-18 combination groups had mean tumor weights that were significantly smaller than those of the empty vector control group (p=0.004-0.038). The results show that not only did the combination of pMUC1/pIL-18 have a positive effect on tumor incidence, it had a positive effect on tumor weights as well. Neither of these effects was observed with either plasmid alone.

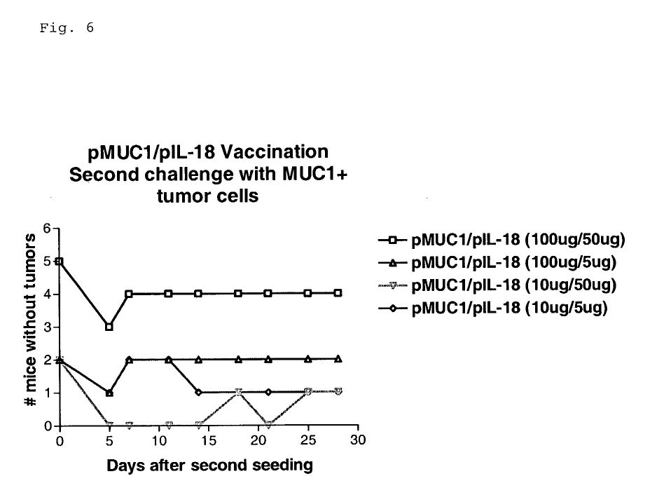

MUC1 Tg mice without tumors from the combination groups were then rechallenged with MUC1⁺ tumor cells on Day 50 (Day 0 in the results shown in FIG. 6) to learn if they had developed protective immunity that could be recalled. Mice were monitored for tumor incidence for 28 days after the second challenge. Of the 5 mice that had originally been vaccinated with 100 ug pMUC1/50 ug pIL-18, 4 of 5 remained free of tumor growths after the second tumor challenge. Both of the mice from the group that was vaccinated with 10 ug pMUC1/5 ug pIL-18 also remained free of growths throughout the second challenge, while 1 of 2 mice each from the two remaining groups developed growths. The results support the hypothesis that the mice developed a memory response that was recalled in response to the second tumor challenge.

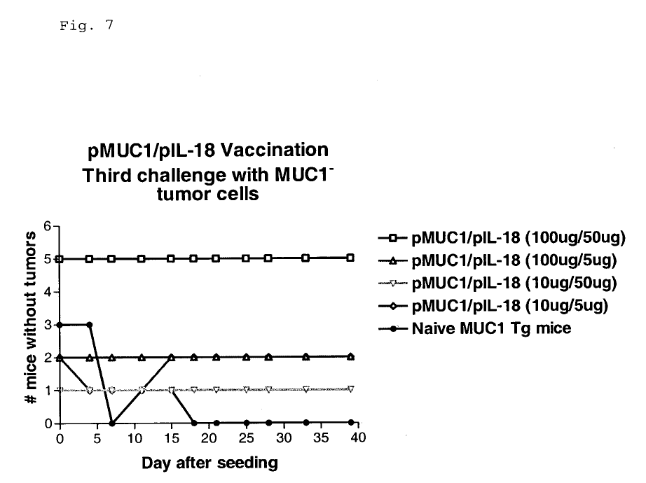

In order to determine if the mice had developed a broader immune response to antigens besides MUC1, the tumor-free MUC1 Tg animals were challenged again but with MUC1⁻ MC38 tumor cells at Day 28 (denoted Day 0 in the results shown in FIG. 7) and monitored for tumor incidence 39 days post challenge. The MC38 cells are the parent line to the MUC1⁺ tumor cells, and are otherwise expected to be identical (Robbins et al., supra).

Interestingly, the mice that were originally vaccinated with the 100 ug dose of pMUC1 in combination with either dose of pIL-18 continue to be protected, while the three naïve control MUC1 Tg mice succumbed to tumors. This result suggests that the vaccinated mice have developed immunity to determinants shared between the two cell lines, in addition to immunity to MUC1, evidencing epitope spreading.

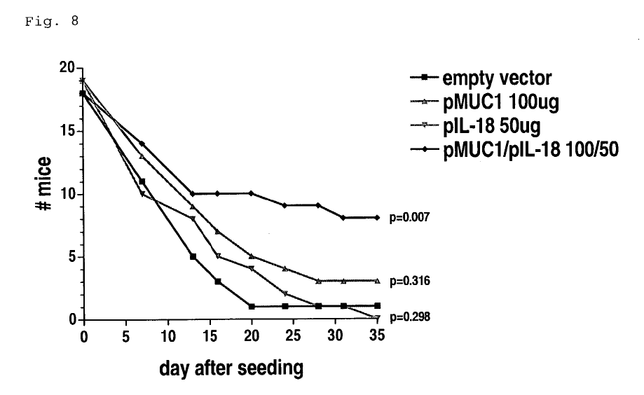

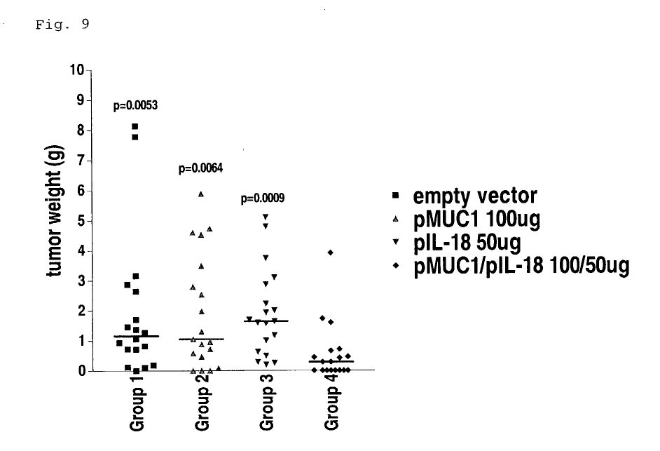

In order to determine if DNA vaccination followed by just a single tumor challenge with MUC1+cells would give rise to epitope spreading, MUC1 Tg mice were vaccinated according to the groups shown in FIG. 8 on Day 0, 14 and 21. Mice were challenged with 1.5×10⁵ MISA cells on Day 28. Vaccination with pMUC1/pIL-18 is the only regimen that results in significant protection (8 of 18 mice) compared to the empty vector group (p=0.007). Median tumor weights are likewise significantly smaller in this group versus the other three groups (FIG. 9). These results confirm the previous data demonstrating that the combination of pMUC1 and pIL-18 offer better protection against tumor challenge and also cause a significant reduction in tumor weight in those animals that still develop tumors. Further, the data indicate that the combination of the two plasmids allows one to break tolerance to the MUC1 self antigen in the MUC1 transgenic mice. The 8 protected mice from the pMUC1/pIL-18 group and the 3 protected mice from the pMUC1 only group were challenged with 3×10⁵ MC38 MUC1⁻ tumor cells 45-47 days after the initial tumor challenge. Only 1 of 15 control naïve animals survived tumor challenge, whereas 4 of 8 and 2 of 3 vaccinated animals remained tumor free. This result indicates that epitope spreading occurs with the immune response generated by the DNA vaccination and the first tumor challenge. Further, the fact that epitope spreading occurs in the pMUC1-only group suggests that IL-18 may not be required for this phenomenon to occur.

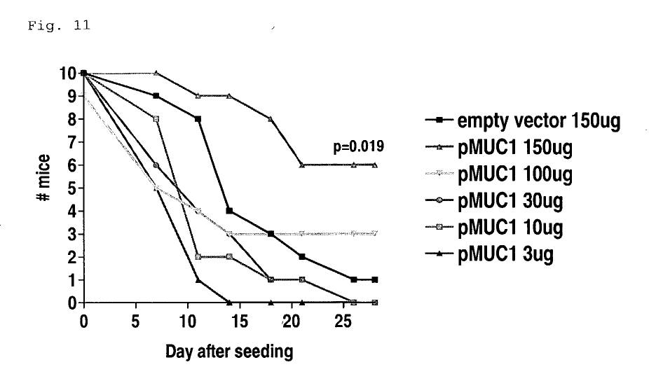

In order to determine whether vaccination with pMUC1 plasmid can induce a protective immune response upon challenge with MISA cells, male MUC1 Tg mice were vaccinated on day 0, 14 and 21 with various doses of DNA, then challenged on Day 28 with 1.5×10⁵ MISA tumor cells (FIG. 11). In the control group, nearly all mice (9 of 10) succumbed to tumors. Male mice vaccinated with 150 ug of pMUC1 showed good protection (6 of 10; p=0.019) and mice vaccinated with 100 ug pMUC1 showed protection in 3 of 9 mice (not significant). Lower doses of pMUC1 did not result in any tumor protection. It appears that the pMUC1 plasmid alone can offer significant benefit in reducing tumor incidence, at high dose.

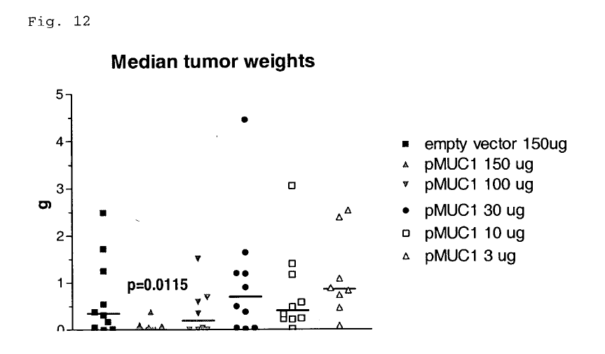

Tumor weights are shown in FIG. 12. Again, the tumor weights in the highest dose group show a significant difference from the control group (p=0.015). This result suggests that the vaccination also helps to control growth of the tumor cells in the mice that still develop tumors.

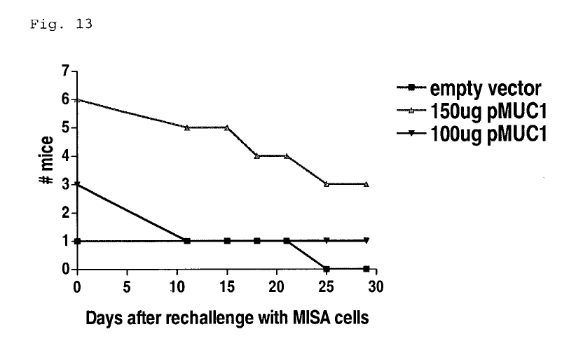

To learn if the anti-tumor response was long-lived, the male mice that did not develop tumors were rechallenged on the opposite flank with 1.5×10⁵ MISA cells on day 39 after the first tumor challenge. As shown in FIG. 13, 3 of 6 and 1 of 3 of the pMUC1 vaccinated mice remained protected after the rechallenge, suggesting that some animals did develop a long-lived recall response to the tumors.

1. A composition comprising a first isolated polynucleotide encoding or complementary to an antigenic determinant of a tumor-associated protein and a second isolated polynucleotide encoding or complementary to a nucleic acid adjuvant.

2. The composition of claim 1 wherein the tumor-associated protein is prostate specific antigen (PSA) or variants thereof.

3. The composition of claim 2 wherein the polynucleotide encoding PSA or variants thereof encodes the amino acid sequences set forth in SEQ ID NOs: 2, 8 or 10.

4. The composition of claim 3 wherein the polynucleotides have the sequence set forth in SEQ ID NOs: 1, 7 or 9.

5. The composition of claim 1 wherein the tumor-associated protein is kallikrein-2 (KLK2) or variants thereof.

6. The composition of claim 5 wherein the first polynucleotide encodes KLK2 or variants thereof having the amino acid sequences set forth in SEQ ID NOs: 24, 27 or 29.

7. The composition of claim 6 wherein the first polynucleotide has the sequence set forth in SEQ ID NOs: 23, 26 or 28.

8. The composition of claim 1 wherein the tumor-associated protein is mucin-1 (MUC1) or variants thereof.

9. The composition of claim 8 wherein the first polynucleotide encodes MUC1 or variants thereof having the amino acid sequences set forth in SEQ ID NOs: 31, 34, 36, 38, 40, 42, 44, 46, 48 or 50.

10. The composition of claim 9 wherein the first polynucleotide has the sequence set forth in SEQ ID NOs: 30, 33, 35, 37, 39, 41, 43, 45, 47 or 49.

11. The composition of claim 1 wherein the nucleic acid adjuvant encodes interleukin-18 (IL-18) or variants thereof.

12. The composition of claim 1 wherein the nucleic acid adjuvant encodes interleukin-12 (IL-12) or variants thereof.

13. The composition of claim 1 wherein the nucleic acid adjuvant encodes granulocyte-macrophage colony-stimulating factor (GM-CSF) or variants thereof.

14. The composition of claim 1 wherein the nucleic acid adjuvant encodes B7-1 or variants thereof.

15. The composition of claim 11 wherein the IL-18 is human and has the amino acid sequence set forth in SEQ ID NO: 64.

16. The composition of claim 15 wherein the human IL-18 is encoded by the nucleotide sequence set forth in SEQ ID NO: 63.

17. The composition of claim 11 wherein the IL-18 variants are human variants and have the amino acid sequence set forth in SEQ ID NOs: 76, 78 or 80.

18. The composition of claim 17 wherein the human IL-18 variants are encoded by the nucleotide sequences set forth in SEQ ID NOs: 75, 77 or 79.

19. The composition of claim 1 further comprising at least one promoter sequence controlling expression of the polynucleotides.

20. A composition comprising a first isolated polynucleotide encoding human PSA having the amino acid sequence set forth in SEQ ID NO: 2, a second isolated polynucleotide encoding a human IL-18 variant having the amino acid sequence set forth in SEQ ID NO: 76 and at least one promoter controlling expression of the polynucleotides.

21. The composition of claim 19 wherein the first polynucleotide has the nucleotide sequence set forth in SEQ ID NO: 1 and the second polynucleotide has the nucleotide sequence set forth in SEQ ID NO: 75.

22. A composition comprising a first isolated polynucleotide encoding human MUC1 having the amino acid sequence set forth in SEQ ID NO: 31, a second isolated polynucleotide encoding a human IL-18 variant having the amino acid sequence set forth in SEQ ID NO: 76 and at least one promoter controlling expression of the polynucleotides.

23. The composition of claim 22 wherein the first polynucleotide has the nucleotide sequence set forth in SEQ ID NO: 30 and the second polynucleotide has the nucleotide sequence set forth in SEQ ID NO: 75.

24. The composition of claims 1, 20 or 22 wherein at least one promoter polynucleotide is human cytomegalovirus immediate early promoter, dihydrofolatereductase promoter, early SV40 promoter or late SV40 promoter.

25. The composition of claims 1, 20 or 22 wherein the first and second polynucleotides are contained on the same nucleic acid vector.

26. The composition of claims 1, 20 or 22 wherein the first and second polynucleotides are contained on separate nucleic acid vectors.

27. The composition of claim 1 further comprising a pharmaceutically acceptable carrier or diluent.

28. A composition comprising a first isolated nucleic acid having the nucleotide sequence shown in SEQ ID NO: 61 and a second isolated nucleic acid having the nucleotide sequence shown in SEQ ID NO: 81.

29. A composition comprising a first isolated nucleic acid having the nucleotide sequence shown in SEQ ID NO: 62 and a second isolated nucleic acid having the nucleotide sequence shown in SEQ ID NO: 81.

30. An isolated nucleic acid comprising a polynucleotide encoding or complementary to an antigenic determinant of PSA, KLK2 or MUC1 and a promoter.

31. The isolated nucleic acid of claim 30 wherein the polynucleotide encodes human PSA having the amino acid sequence set forth in SEQ ID NO: 2.

32. The isolated nucleic acid of claim 31 having the polynucleotide sequence set forth in SEQ ID NO: 62.

33. The isolated nucleic acid of claim 30 wherein the polynucleotide encodes human MUC1 having the amino acid sequence set forth in SEQ ID NO: 31.

34. The isolated nucleic acid of claim 33 having the polynucleotide sequence set forth in SEQ ID NO: 61.

35. A method for eliciting an immune response to a cancer associated tumor protein in a mammal that is prophylactic or therapeutic for the cancer comprising administering the composition of claim 1 to the mammal.

36. A method for eliciting an immune response to PSA in a mammal that is prophylactic or therapeutic for prostate cancer comprising administering the composition of claim 20 to the mammal.

37. A method for eliciting an immune response to PSA in a mammal that is prophylactic or therapeutic for prostate cancer comprising administering the isolated nucleic acid of claim 29 to the mammal.

38. A method for eliciting an immune response to MUC1 in a mammal that is prophylactic or therapeutic for prostate or breast cancer comprising administering the composition of claim 22 to the mammal.

39. A method for eliciting an immune response to MUC1 in a mammal that is prophylactic or therapeutic for prostate or breast cancer comprising administering the composition of claim 28 to the mammal.

40. A method for eliciting an immune response to a cancer associated tumor protein in a mammal for therapy of a tumor-associated pathology comprising administering to the mammal the composition of claim 1.


SEQUENCE LISTING (81 sequences)

SEQ ID NO: 1 - DNA, 967 bp, Homo sapiens
  CDS (1)...(810)
ctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcacacacccgctctacgatatgagcctcctgaagaatcgattcctcaggccaggtgatgactccagccacgacctcatgctgctccgcctgtcagagcctgccgagctcacggatgctatgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaactccctattgtagtaaacttggaaccttggaaatgaccaggccaagactcaggcctccccagttctactgacctttgtccttaggtgtgaggtccagggttgctaggaaaagaaatcagcagacacaggtgtagaccagagtgt

SEQ ID NO: 2 - protein, 269 aa, Homo sapiens
LHPESCVTMWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFTHPLYDMSLLKNRFLRPGDDSSHDLMLLRLSEPAELTDAMKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 3 - DNA, 1394 bp, Homo sapiens
gtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaaccccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatactgtg

SEQ ID NO: 4 - DNA, 1729 bp, Homo sapiens
aagtttcccttctcccagtccaagaccccaaatcaccacaaaggacccaatccccagactcaagatatggtctgggcgctgtcttgtgtctcctaccctgatccctgggttcaactctgctcccagagcatgaagcctctccaccagcaccagccaccaacctgcaaacctagggaagattgacagaattcccagcctttcccagctccccctgcccatgtcccaggactcccagccttggttctctgcccccgtgtcttttcaaacccacatcctaaatccatctcctatccgagtcccccagttcctcctgtcaaccctgattcccctgatctagcaccccctctgcaggtgctgcacccctcatcctgtctcggattgtgggaggctgggagtgcgagaagcattcccaaccctggcaggtgcttgtagcctctcgtggcagggcagtctgcggcggtgttctggtgcacccccagtgggtcctcacagctacccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctatgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaactccctattgtagtaaacttggaaccttggaaatgaccaggccaagactcaggcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgaaccaaggagggagggtcttcctttggcatgggatggggatgaagtaaggagagggactgaccccctggaagctgattcactatggggggaggtgtattgaagtcctccagacaaccctcagatttgatgatttcctagtagaactcacagaaataaagagctgttatactgtgaa

SEQ ID NO: 5 - DNA, 711 bp, Macaca mulatta
attgtgggaggctgggagtgcgagaagcattcccaaccctggcaggtgcttgtggcctctcgtggcagggcagtctgcgggggtgttctggtgcacccccagtgggtcctcacagctgcccactgcatcaggagcaacagcgtgatcttgctgggtcggcacaacccgtattatcctgaagacacgggccaggtgtttcaggtcagccacagcttcccacacccgctctacaacatgagcctcctgaagaatcgatacctcgggccaggtgatgactccagccacgacctcatgctgctccgcctgtcagagcctgccgagatcacagatgctgtgcaggtcctggacctgcccacctgggagccagagctggggaccacgtgctacgcctcaggctggggcagcatcgaaccggaggaacacttgactccaaagaaacttcagtgtgtggacctccatattatttccaatgatgtgtgtgcgcaagttcactctcagaaggtgaccaagttcatgctgtgtgctggaagctggatgggcggcaaaagcacctgctcgggtgattctgggggcccactggtctgtgacggtgtgcttcaaggtatcacgtcatggggcagtcaaccatgtgccctaccccgaaggccttccctgtacaccaaggtggtgcgttaccggaagtggatccaggacaccatcatggcaaacccc

SEQ ID NO: 6 - protein, 237 aa, Macaca mulatta
IVGGWECEKHSQPWQVLVASRGRAVCGGVLVHPQWVLTAAHCIRSNSVILLGRHNPYYPEDTGQVFQVSHSFPHPLYNMSLLKNRYLGPGDDSSHDLMLLRLSEPAEITDAVQVLDLPTWEPELGTTCYASGWGSIEPEEHLTPKKLQCVDLHIISNDVCAQVHSQKVTKFMLCAGSWMGGKSTCSGDSGGPLVCDGVLQGITSWGSQPCALPRRPSLYTKVVRYRKWIQDTIMANP

SEQ ID NO: 7 - DNA, 1654 bp, Homo sapiens
ggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgtgggtcattctgatcaccgaactgaccatgccagccctgccgatggtcctccatggctccctagtgccctggagaggaggtgtctagtcagagagtagtcctggaaggtggcctctgtgaggagccacggggacagcatcctgcagatggtcctggcccttgtcccaccgacctgtctacaaggactgtcctcgtggaccctcccctctgcacaggagctggaccctgaagtcccttccctaccggccaggactggagcccctacccctctgttggaatccctgcccaccttcttctggaagtcggctctggagacatttctctcttcttccaaagctgggaactgctatctgttatctgcctgtccaggtctgaaagataggattgcccaggcagaaactgggactgacctatctcactctctccctgcttttacccttagggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaactccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatgggttgtctgtgttatttgtggggtacagagatgaaagaggggtggggatccacactgagagagtggagagtgacatgtgctggacactgtccatgaagcactgagcagaagctggaggcacaacgcaccagacactcacagcaaggatggagctgaaaacataacccactctgtcctggagg

SEQ ID NO: 8 - protein, 234 aa, Homo sapiens
VVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSWVILITELTMPALPMVLHGSLVPWRGGV

SEQ ID NO: 9 - DNA, 658 bp, Homo sapiens
  CDS (2)...(529)
ggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagtgtacgcctgggccagatggtgcagccgggagcccagatgcctgggtctgagggaggaggggacaggactcctgggtctgagggaggagggccaaggaaccaggtggggtccagcccacaacagtgttttttgcctggcccgtagtcttgaccccaaagaaacttcagtgtgtggac

SEQ ID NO: 10 - protein, 176 aa, Homo sapiens
VVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEECTPGPDGAAGSPDAWV

SEQ ID NO: 11 - DNA, 585 bp, Homo sapiens
  CDS (1)...(585)
attgtgggaggctgggagtgcgagaagcattcccaaccctggcaggtgcttgtggcctctcgtggcagggcagtctgcggcggtgttctggtgcacccccagtgggtcctcacagctgcccactgcatcaggaagccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctga

SEQ ID NO: 12 - protein, 194 aa, Homo sapiens
IVGGWECEKHSQPWQVLVASRGRAVCGGVLVHPQWVLTAAHCIRKPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 13 - DNA, 30 bp, Homo sapiens
  CDS (1)...(30)
ttcttgaccccaaagaaacttcagtgtgtg

SEQ ID NO: 14 - protein, 10 aa, Homo sapiens
FLTPKKLQCV

SEQ ID NO: 15 - DNA, 27 bp, Homo sapiens
  CDS (1)...(27)
aaacttcagtgtgtggacctccatgtt

SEQ ID NO: 16 - protein, 9 aa, Homo sapiens
KLQCVDLHV

SEQ ID NO: 17 - DNA, 30 bp, Homo sapiens
  CDS (1)...(30)
gttatttccaatgacgtgtgtgcgcaagtt

SEQ ID NO: 18 - protein, 10 aa, Homo sapiens
VISNDVCAQV

SEQ ID NO: 19 - DNA, 27 bp, Homo sapiens
  CDS (1)...(27)
gttctggtgcacccccagtgggtcctc

SEQ ID NO: 20 - protein, 9 aa, Homo sapiens
VLVHPQWVL

SEQ ID NO: 21 - DNA, 27 bp, Homo sapiens
  CDS (1)...(27)
caagttcaccctcagaaggtgaccaag

SEQ ID NO: 22 - protein, 9 aa, Homo sapiens
QVHPQKVTK

SEQ ID NO: 23 - DNA, 711 bp, Homo sapiens
  CDS (1)...(711)
attgtgggaggctgggagtgtgagaagcattcccaaccctggcaggtggctgtgtacagtcatggatgggcacactgtgggggtgtcctggtgcacccccagtgggtgctcacagctgcccattgcctaaagaagaatagccaggtctggctgggtcggcacaacctgtttgagcctgaagacacaggccagagggtccctgtcagccacagcttcccacacccgctctacaatatgagccttctgaagcatcaaagccttagaccagatgaagactccagccatgacctcatgctgcttcgcctgtcagagcctgccaagatcacagatgttgtgaaggtcctgggcctgcccacccaggagccagcactggggaccacctgctacgcctcaggctggggcagcatcgaaccagaggagttcttgcgccccaggagtcttcagtgtgtgagcctccatctcctgtccaatgacatgtgtgctagagcttactctgagaaggtgacagagttcatgttgtgtgctgggctctggacaggtggtaaagacacttgtgggggtgattctgggggtccacttgtctgtaatggtgtgcttcaaggtatcacatcatggggccctgagccatgtgccctgcctgaaaagcctgctgtgtacaccaaggtggtgcattaccggaagtggatcaaggacaccatcgcagccaacccc

SEQ ID NO: 24 - protein, 237 aa, Homo sapiens
IVGGWECEKHSQPWQVAVYSHGWAHCGGVLVHPQWVLTAAHCLKKNSQVWLGRHNLFEPEDTGQRVPVSHSFPHPLYNMSLLKHQSLRPDEDSSHDLMLLRLSEPAKITDVVKVLGLPTQEPALGTTCYASGWGSIEPEEFLRPRSLQCVSLHLLSNDMCARAYSEKVTEFMLCAGLWTGGKDTCGGDSGGPLVCNGVLQGITSWGPEPCALPEKPAVYTKVVHYRKWIKDTIAANP

SEQ ID NO: 25 - DNA, 6139 bp, Homo sapiens
gctggatgtggtggtgcatgcttgtggtctcagctatcctggaggctgagacaggagaatcggttgagtctgggagttcaaggctacagggagctgcgatcacgccgctgcactccagcctgggaaacagagtgagactgtctcagaatttttttaaaaaagaatcagtgatcatcccaacccctgttgctgttcatcctgagcctgccttctctggctttgttccctagatcacatctccatgatccataggccctgcccaatctgacctcacaccgtgggaatgcctccagactgatctagtatgtgtggaacagcaagtgctggctctccctccccttccacagctctgggtgtgggagggggttgtccagcctccagcagcatggggagggccttggtcagcatctaggtgccaacagggcaagggcggggtcctggagaatgaaggctttatagggctcctcagggaggccccccagccccaaactgcaccacctggccgtggacacctgtgtcagcatgtgggacctggttctctccatcgccttgtctgtggggtgcactggtgagattggggggataaaggaaggggggcgggttctgactcttatgctgaagcccttttcctcccacccagtgccccagcctcgtcccttcagcccacagttcagcccagacaatgtgcccctgactcttccacattgcaatagtcctcatgcccacactaggtccccgctccctcccacttacctcagacctttctctccattgcccagccaaatccctgctcccagctgctttactaaagagcaagttcctaggcatctctgtgtttctctttatggggttcaaaacctttcaaggacctctctccatgccactggttccttggaccctatcactgggctgcctcctgagcccctcagtcctaccacagtctactgacttttcccattcagctgtgagcattcaaccctgtcccctggaccttgacacctggctccccaaccctgtcccaggaaacccagattccaccagacacttccttcttcccccccgaggctatctggcctgagacaacaaatgctgcctcccaccctgagtctggcactgggactttcagaactcctccttccctgactctttgccccagacccgtcattcaatggctagctttttccatgggaagaagaacaacgagcacccccaaccacaacggccagttctctgattccctaaatccgcacccttttcaaaacctcaaaaacaaaacaaaacaaaacaaagcaagaaacaactcaggcaaaacttgttgcttaaccttggacatggtaaaccatccaaaaccttcctctcccagcaactaaacctctccactgggcacttaacctttggtttcttggaacctcttaatctcttagaacccacagctgccaccacatgcccttctcccaatgtaagaccccaaatcactccaaatgacccaacccccaacccatgcctccttcagatatttcccatgtcccctactctgatctctggggtcagctccgttctcgagagcatgaagcctcccgacctggtccagccaccaacccgctaacgcagggaatagctacagaattgccagccctcccaggaccccttgcttgtgtcctggactcccagtcctggtcctctgcccccatgtctcttcaaacccacagctcagctccctcccctatccaattcttttgggtctgatccccctgacccagcaccccctccgcaggtgccgtgcccctcatccagtctcggattgtgggaggctgggagtgtgagaagcattcccaaccctggcaggtggctgtgtacagtcatggatgggcacactgtgggggtgtcctggtgcacccccagtgggtgctcacagctgcccattgcctaaagaagtaagtaggaccctgggatctggggagggaatggctgtgtcccacaggaataacagcgggatgcttcccccagggtcacttctcaggtgaggcttcagactaaaggagagagggaaggtcctggcccaggtcgcacccggaggcagagctggggctggaccactctccccatggctgcctgggtttctctctgtgtctgatctcgctgtgtctcttggtatctggctctggttgtgtctgtatgactgtgttttggtctctatgtccctctctcttttctgtctccctgtgtctgtgtctcccccgtctctgtctctgggtctctctgtggccatctctgtcaccgtgtgtctcaccctgcatctctttgcctgtctttctctctgggtctctgcctcagcccttcctcatcactactgaacacaccccgtgaggtgggtggggagcacccagaaaaaggaaggactttaagctcaatgtgtgtgcatgtgagggggtgcctgtcattgcacagcactctctgcaggacatccctccaccctggggagacacagggagggctggtttcagctgtagctgggtgcacagttgaggagggaggaaggagaaggggaaacaagaaaggaggggaaggtggccgggcacggtggcccacgcctgtaatcccagcactttgggaggccgaggtgggtggatcatctgaggtcaggagtttgaaaccagcctggccaacatggcaaaaccccgtctctactaaaaatacaaaaagtagccaggcgtggtgctgcgcgcctgtaatccaattactagggaggctgaggcaggagaatcgcttgaacccgggaggcagaggttgcagtgagccgagatcgtgccactgcactccagcctgggtgacagagcaagactccatctcagaaaaaacaaacaaacaaacaaacaacaaaaaaaatcgaaaggaggggaagggagctggagagagaaagggggacatggccctgagctgtgggccgggccacccgccactacagagccctcactccagccccagctgcaggtgagccaccctcatgcctctcctcctccccctgctactccacactcctcagatgcccccgtggcctccctcctttttctctcccacactgtatcacccctggcttcctctctgctgtttctccttctctctctgacttcccgcatccttttctcatttgtctatttctcactcccttcctggttctgttctttctcccttcctcttccccatgtctatttcttgctgtctctgtctcttctttgctcatcctaattctcactgttctcccttctgtttttgtcattcctctgccattttatgctctctcttttccacttcgtttctttcagtttctgtctctgcctctcacatgatcacactcctgttttctaactcactgtctgtatttcaccacgactatatctccccgacccctgtgcttttctcactgtttctttttcttccctttggagtctcccttatcctcccctgccccatctacctttccccattttctctctcctcatgcatccacccccttcctccccaggaatagccaggtctggctgggtcggcacaacctgtttgagcctgaagacacaggccagagggtccctgtcagccacagcttcccacacccgctctacaatatgagccttctgaagcatcaaagccttagaccagatgaagactccagccatgacctcatgctgctccgcctgtcagagcctgccaagatcacagatgttgtgaaggtcctgggcctgcccacccaggagccagcactggggaccacctgctacgcctcaggctggggcagcatcgaaccagaggagtgtacgcctgggccagatggtgtagctgggagcccagatgcctgggtctgagggaagtggggccaaagaaccaggtggggtccggccacagcccagtttttctctgacccatagtcttgcgccccaggagtcttcagtgtgtgagcctccatctcctgtccaatgacatgtgtgctagagcttactctgagaaggtgacagagttcatgttgtgtgctgggctctggacaggtggtaaagacacttgtggggtgagtcatccctactcccaacatctggaggggaaaggtgagtgaagaccctaattctgggctgcaatctgaaagctaaccagacatctgcctcccctgctccccagctatagccacgccccctccccatgcctcatctgccgccctccttcccccttccctgactccctcaacacaagaggtgattctcacagcataattcacccattcctgtgttgagcacatgcttactgggcactgctacgtgaccagcattgccgtagaccctgggaagcagcagtgaacaggtagagagcagcctctccctcctgcagcccccatgctggtgaggggcactggcaggaacagtggacccaacatggaaatgctggagggtgtcaggaagtgatcgggctctggggcagggaggaggggtggggagtgtcactgggaggggacatcctgcagaaggtaggagtgagcaaacacccgctgcaggggaggggagagccctgcggcacctgggggagcagagggagcagcacctgcccaggcctgggaggaggggccgggagggcgtgaggaggagcgagggggctgcatggctggagtgagggatcaggggcagggcgcgagatggcctcacacagggaagagagggcccctcctgcagggcctcacctgggccacaggaggacactgcttttcctctgaggagtcaggagctgtggatggtgctggacagaagaaggacagggcctggctcaggtgtccagaggctgtcgctggcttccctttgggatcagactgcagggagggagggcggcagggttgtggggggagtgacgatgaggatgacctgggggtggctccaggccttgcccctgcctgggccctcacccagcctccctcacagtctcctggccctccagtctctcccctccactccatcctccatctggcctcagtgggtcattctgatcactgaactgaccatacccagccctgcccacggccctccatggctccccaatgccctggagaggggacatctagtcagagagtagtcctgaagaggtggcctctgcgatgtgcctgtgggggcagcaacctgcagatggtcccggccctcatcctgctgacctgtctgcagggatgtcctcctggaccttgcccctgtgcaggagctggaccctgaagtcccctccccataggccaagactggagccttgttccctctgttggactccctgcccatattcttgtgggagtgggttctggagacatttctgtctgttcctgagagctgggaattgctctcagtcatctgcctgcgcggttctgagagatggagttgcctaggcagttattggggccaatctttctcactgtgtctctcctcctttacccttagggtgattctgggggtccacttgtctgtaatggggtgcttcaaggtatcacatcatggggccctgagccatgtgccctgcctgaaaagcctgctgtgtacaccaaggtggtgcattaccggaagtggatcaaggacaccatcgcagccaacccctgagtgcccctgtcccacccctacctctagtaaatttaagtccacctcacgttctggcatcacttggcctttctggatgctggacacctgaagcttggaactcacctggccgaagctcgagcctcctgagtcctactgacctgtgctttctggtgtggagtccagggctgctaggaaaaggaatgggcagacacaggtgtatgccaatgtttctgaaatgggtataatttcgtcctctccttcggaacactggctgtctctgaagacttctcgctcagtttcagtgaggacacacacaaagacgtgggtgaccatgttgtttgtggggtgcagagatgggaggggtggggcccacctggaagagtggacagtgacacaaggtggacactctctacagatcactgaggataagctggagccacaatgcatgaggcacacacacagcaaggatgacgctgtaaacatagcccacgctgtcctgggggcactgggaagcctagataaggccgtgagcagaaagaaggggaggatcc

SEQ ID NO: 26 - DNA, 420 bp, Homo sapiens
  CDS (1)...(420)
attgtgggaggctgggagtgtgagaagcattcccaaccctggcaggtggctgtgtacagtcatggatgggcacactgtgggggtgtcctggtgcacccccagtgggtgctcacagctgcccattgcctaaagaagaatagccaggtctggctgggtcggcacaacctgtttgagcctgaagacacaggccagagggtccctgtcagccacagcttcccacacccgctctacaatatgagccttctgaagcatcaaagccttagaccagatgaagactccagccatgacctcatgctgcttcgcctgtcagagcctgccaagatcacagatgttgtgaaggtcctgggcctgcccacccaggagccagcactggggaccacctgctacgcctcaggctggggcagcatcgaaccagaggag

SEQ ID NO: 27 - protein, 140 aa, Homo sapiens
IVGGWECEKHSQPWQVAVYSHGWAHCGGVLVHPQWVLTAAHCLKKNSQVWLGRHNLFEPEDTGQRVPVSHSFPHPLYNMSLLKHQSLRPDEDSSHDLMLLRLSEPAKITDVVKVLGLPTQEPALGTTCYASGWGSIEPEE

SEQ ID NO: 28 - DNA, 597 bp, Homo sapiens
  CDS (1)...(597)
attgtgggaggctgggagtgtgagaagcattcccaaccctggcaggtggctgtgtacagtcatggatgggcacactgtgggggtgtcctggtgcacccccagtgggtgctcacagctgcccattgcctaaagaagaatagccaggtctggctgggtcggcacaacctgtttgagcctgaagacacaggccagagggtccctgtcagccacagcttcccacacccgctctacaatatgagccttctgaagcatcaaagccttagaccagatgaagactccagccatgacctcatgctgctccgcctgtcagagcctgccaagatcacagatgttgtgaaggtcctgggcctgcccacccaggagccagcactggggaccacctgctacgcctcaggctggggcagcatcgaaccagaggagttcttgcgccccaggagtcttcagtgtgtgagcctccatctcctgtccaatgacatgtgtgctagagcttactctgagaaggtgacagagttcatgttgtgtgctgggctctggacaggtggtaaagacacttgtggggtgagtcatccctactcccaacatctggaggggaaaggg

SEQ ID NO: 29 - protein, 199 aa, Homo sapiens
IVGGWECEKHSQPWQVAVYSHGWAHCGGVLVHPQWVLTAAHCLKKNSQVWLGRHNLFEPEDTGQRVPVSHSFPHPLYNMSLLKHQSLRPDEDSSHDLMLLRLSEPAKITDVVKVLGLPTQEPALGTTCYASGWGSIEPEEFLRPRSLQCVSLHLLSNDMCARAYSEKVTEFMLCAGLWTGGKDTCGVSHPYSQHLEGKG

SEQ ID NO: 30 - DNA, 921 bp, Homo sapiens
  CDS (1)...(921)
atgacaccgggcacccagtctcctttcttcctgctgctgctcctcacagtgcttacagttgttacaggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccagggtgtcacctcggccccggagaccaggccgcccccgggctccaccgcccccccagcccatggtgtcacctcggcgccggacaacaggcccgccttggcgtccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatggtgagtatcggcctttccttccccatgctcccc

SEQ ID NO: 31 - protein, 307 aa, Homo sapiens
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAQGVTSAPETRPPPGSTAPPAHGVTSAPDNRPALASTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMVSIGLSFPMLP

SEQ ID NO: 32 - DNA, 3343 bp, Homo sapiens
gagctcctggccagtggtggagagtggcaaggaaggaccctagggttcatcggagcccaggtttactcccttaagtggaaatttcttcccccactcccctccttggctttctccaaggagggaaccccaggctgctggaaagtccggctggggcggggactgtgggtttcagggtagaactgcgtgtggaacgggacagggagcggttagaagggtggggctattccgggaagtggtggtggggggagggagcccaaaactagcacctagtccactcattatccagccctcttatttctcggccgcctctgcttcagtggacccggggagggcggggaagtggagtgggagacctaggggtgggcttcccgaccttgctgtacaggacctcgacctagctggctttgttccccatccccagttagttgttgccctgaggctaaaactagagcccaggggccccaagttccagactgcccctcccccctcccccggagccagggagtggttggtgaaagggggaggccagctggagaagaaacgggtagtcaggggttgcagcattagagcccttgtagccctagcccaggaatggttggagagagaagagtagagtagggaggggggtttgtcacctgtcacctgctcggctgtgcctagggcgggcgggggggagtggggggaccggtataaagcggtaggcgcctgtgcccgctccacctctcaagcagccagcgcctgcctgaatctgttctgccccctccccacccatttcaccaccaccatgacaccgggcacccagtctcctttcttcctgctgctgctcctcacagtgcttacaggtgaggggcacgaggtggggagtgggctgccctgcttaggtggtcttcgtggtctttctgtgggttttgctccctggcagatggcaccagaagttaaggtaagaattgcagacagaggctgccctgtctgtgccagaaggagggagaggctaaggacaggctgagaagagttgcccccaaccctgagagtgggtaccaggggcaagcaaatgtcctgtagagaagtctagggggaagagagtagggagagggaaggcttaagaggggaagaaatgcaggggccatgagccaaggcctatgggcagagagaaggaggctgctgcaggaaggaggcggccaacccaggggttactgaggctgcccactccccagtcctcctggtattatttctctggtggccaggcttatattttcttcttgctcttatttttccttcataaagacccaaccctatgactttaacttcttacagctaccacagcccctgggcccgcaacagttgttacaggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccagggtgtcacctcggccccggagaccaggccgcccccgggctccaccgcccccccagcccatggtgtcacctcggcgccggacaacaggcccgccttggcgtccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatggtgagtatcggcctttccttccccatgctcccctgaagcagccatcagaactgtccacaccctttgcatcaagcctgagtcctttccctctcaccccagtttttgcagatttataaacaagggggttttctgggcctctccaatattaagttcaggtacagttctgggtgtggacccagtgtggtggttggaggggtgggtggtggtcatgagccgtagggagggactggtgcacttaaggttgggggaagagtgctgagccagagctgggacccgtggctgaagtgcccatttccctgtgaccaggccaggatctgtggtggtacaattgactctggccttccgagaaggtaccatcaatgtccacgacgtggagacacagttcaatcagtataaaacggaagcagcctctcgatataacctgacgatctcaagacgtcagcggtgaggctacttccctgctgcagccagcaccatgccggggcccctctccttccagtgtctgggtccccgctctttccttagtgctggcagcgggaggggcgcctcctctgggagactgccctgaccactgcttttccttttagtgagtgatgtgccatttcctttctctgaccagtctggggctggggtgccaggctggggcatcgcgctgctggtgctggtctgtgttctggttgcgctggccattgtctatctcattgccttggtgagtgcagtccctggccctgatcagagccccccggtagaaggcactccatggcctgccataacctcctatctccccaggctgtctgtcagtgccgccgaaagaactacgggcagctggacatctttccagcccgggatacctaccatcctatgagcgagtaccccacctaccacacccatgggcgctatgtgcccctagcagtaccgatcgtagcccctatgagaaggtgagattgggccccacaggcaggggaagcagagggtttggctgggcaaggattctgaagggggtacttggaaaacccaaagagcttggaagaggtgagaagtggcgtgaagtgagcaggggagggctggcaaggatgaggggcagaggtcagaggagttttgggggacaggcctgggaggagactatggaagaaaggggcccctcaaaagggagtgccccactgccagaattc

SEQ ID NO: 33 - DNA, 765 bp, Homo sapiens
  CDS (1)...(765)
atgacaccgggcacccagtctcctttcttcctgctgctgctcctcacagtgcttacagttgttacaggttctggtcatgcaagctctaccccaggtggagaaaaggagacttcggctacccagagaagttcagtgcccagctctactgagaagaatgcttttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttg

SEQ ID NO: 34 - protein, 255 aa, Homo sapiens
MTPGTQSPFFLLLLLTVLTVVTGSGHASSTPGGEKETSATQRSSVPSSTEKNAFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 35 - DNA, 504 bp, Homo sapiens
  CDS (1)...(504)
atgacaccgggcacccagtctcctttcttcctgctgctgctcctcacagtgcttacagttgttacaggttctggtcatgcaagctctaccccaggtggagaaaaggagacttcggctacccagagaagttcagtgcccagctctactgagaagaatgctttgtctactggggtctctttctttttcctgtcttttcacatttcaaacctccagtttaattcctctctggaagatcccagcaccgactactaccaagagctgcagagagacatttctgaaatggctgtctgtcagtgccgccgaaagaactacgggctgctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttg

SEQ ID NO: 36 - protein, 168 aa, Homo sapiens [sequence omitted]

SEQ ID NO: 37 - DNA, 756 bp, Homo sapiens
  CDS (1)...(756)
atgacaccgggcacccagtctcctttcttcctgctgctgctcctcacagtgcttacaggttctggtcatgcaagctctaccccaggtggagaaaaggagacttcggctacccagagaagttcagtgcccagctctactgagaagaatgcttttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacatggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttg

SEQ ID NO: 38 - protein, 252 aa, Homo sapiens [sequence omitted]

SEQ ID NO: 39 - DNA, 231 bp, Homo sapiens
  CDS (1)...(231)
atgacaccgggcacccagtctcctttcttcctgctgctgctcctcacagtgcttacagttgttacaggttctggtcatgcaagctctaccccaggtggagaaaaggagacttcggctacccagagaagttcagtgcccagctctactgagaagaatgctttgtctactggggtctctttctttttcctgtcttttcacatttcaaacctccagtttaattcctctctggaa

SEQ ID NO: 40 - protein, 77 aa, Homo sapiens
MTPGTQSPFFLLLLLTVLTVVTGSGHASSTPGGEKETSATQRSSVPSSTEKNALSTGVSFFFLSFHISNLQFNSSLE

SEQ ID NO: 41 - DNA, 327 bp, Homo sapiens
  CDS (1)...(327)
atgacaccgggcacccagtctcctttcttcctgctgctgctcctcacagtgcttacagctaccacagcccctaaacccgcaacagttgttacaggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcg

SEQ ID NO: 42 - protein, 109 aa, Homo sapiens
MTPGTQSPFFLLLLLTVLTATTAPKPATVVTGSGHASSTPGGEKETSATQRSSVPSSTEKNAVSMTSSVLSSHSPGSGSSTTQGQDVTLAPATEPASGSAATWGQDVTS

SEQ ID NO: 43 - DNA, 468 bp, Homo sapiens
  CDS (1)...(468)
atgacaccgggcacccagtctcctttcttcctgctgctgctcctcacagtgcttacagctaccacagcccctaaacccgcaacagttgttacaggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccacggtgtcacctcggccccggacaccaggccggcc

SEQ ID NO: 44 - protein, 156 aa, Homo sapiens
MTPGTQSPFFLLLLLTVLTATTAPKPATVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPA

SEQ ID NO: 45 - DNA, 129 bp, Homo sapiens
  CDS (1)...(129)
atgacaccgggcacccagtctcctttcttcctgctgctgctcctcacagtgcttacaggttctggtcatgcaagctctaccccaggtggagaaaaggagacttcggctacccagagaagttcagtgccc

SEQ ID NO: 46 - protein, 43 aa, Homo sapiens
MTPGTQSPFFLLLLLTVLTGSGHASSTPGGEKETSATQRSSVP

SEQ ID NO: 47 - DNA, 102 bp, Homo sapiens
  CDS (1)...(102)
atgacaccgggcacccagtctcctttcttcctgctgctgctcctcacagtgcttacaggtggagaaaaggagacttcggctacccagagaagttcagtgccc

SEQ ID NO: 48 - protein, 34 aa, Homo sapiens
MTPGTQSPFFLLLLLTVLTGGEKETSATQRSSVP

SEQ ID NO: 49 - DNA, 204 bp, Homo sapiens
  CDS (74)...(202)
ccgctccacctctcaagcagccagcgcctgcctgaatctgttctgccccctccccacccatttcaccaccaccatgacaccgggcacccagtctcctttcttcctgctgctgctcctcacagtgcttacaggttctggtcatgcaagctctaccccaggtggagaaaaggagacttcggctacccagagaagttcagtgcccag

SEQ ID NO: 50 - protein, 43 aa, Homo sapiens; identical to SEQ ID NO: 46

SEQ ID NO: 51 - DNA, 27 bp, Homo sapiens
  CDS (1)...(27)
tccaccgcccctccagtccacaatgtc

SEQ ID NO: 52 - protein, 9 aa, Homo sapiens
STAPPVHNV

SEQ ID NO: 53 - DNA, 27 bp, Homo sapiens
  CDS (1)...(27)
ctgctgctcctcacagtgcttacagtt

SEQ ID NO: 54 - protein, 9 aa, Homo sapiens
LLLLTVLTV

SEQ ID NO: 55 - DNA, 27 bp, Homo sapiens
  CDS (1)...(27)
tccaccgcccccccagcccatggtgtc

SEQ ID NO: 56 - protein, 9 aa, Homo sapiens
STAPPAHGV

SEQ ID NO: 57 - DNA, 21 bp, Homo sapiens
  CDS (1)...(21)
gcgccggacactaggcccgcc

SEQ ID NO: 58 - protein, 7 aa, Homo sapiens
APDTRPA

SEQ ID NO: 59 - DNA, 36 bp, Homo sapiens
  CDS (1)...(36)
ccgggctccaccgcccccccagcccatggtgtcacc

SEQ ID NO: 60 - protein, 12 aa, Homo sapiens
PGSTAPPAHGVT

SEQ ID NO: 61 - DNA, 6245 bp, Artificial Sequence
  promoter (2767)...(3521): HCMV\ promoter
gatccgcgttgcggccgcaaaaagtcgacgggcgacgcgtaaaaagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccggcccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatagccgcggcatcgatgatatcgcggctatctgaggggactagggtgtgtttaggcgaaaagcggggcttcggttgtacgcggttaggagtcccctcaccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacctccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagcttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttagatctgtctagaagctgggtaccagctgctagccaccatgacaccgggcacccagtctcctttcttcctgctgctgctcctcacagtgcttacagctaccacagcccctaaacccgcaacagttgttacgggttctggtcatgcaagctctaccccaggtggagaaaaggagacttcggctacccagagaagttcagtgcccagctctactgagaagaatgctgtgagtatgacaagcttgatatcgaattccggtgtccggggctccaccggccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgaacccccagcccacggtgtcacctcggccccggacaccaggcgggccccgggctccaccccggccccgggctccaccgcccccccagcccacggtgtcacctcggccccggacaccaggccggccccgggctccaccgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatcccagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactagcccagagagccctgagatagcggg

SEQ ID NO: 62 - DNA, 4775 bp, Artificial Sequence
  CDS (1)...(1177): Kanamycin resistance
aaatgggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgcgttcaaaatggtatgcgttttgacacatccactatatatccgtgtcgttctgtccactcctgaatcccattccagaaattctctagcgattccagaagtttctcagagtcggaaagttgaccagacattacgaactggcacagatggtcataacctgaaggaagatctgattgcttaactgcttcagttaagaccgacgcgctcgtcgtataacagatgcgatgatgcagaccaatcaacatggcacctgccattgctacctgtacagtcaaggatggtagaaatgttgtcggtccttgcacacgaatattacgccatttgcctgcatattcaaacagctcttctacgataagggcacaaatcgcatcgtggaacgtttgggcttctaccgatttagcagtttgatacactttctctaagtatccacctgaatcataaatcggcaaaatagagaaaaattgaccatgtgtaagcggccaatctgattccacctgagatgcataatctagtagaatctcttcgctatcaaaattcacttccaccttccactcaccggttgtccattcatggctgaactctgcttcctctgttgacatgacacacatcatctcaatatccgaatagggcccatcagtctgacgaccaagagagccataaacaccaatagccttaacatcatccccatatttatccaatattcgttccttaatttcatgaacaatcttcattctttcttctctagtcattattattggtccgttcataacaccccttgtattactgtttatgtaagcagacagttttattgttcatgatgatatatttttatcttgtgcaatgtaacatcagagattttgagacacaacgtggctttccccggcccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatagccgcggctatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaagcttcgaccaattctcatgtttgacagcttatcatcgcagatccgggcaacgttgttgccattgctgcaggcgcagaactggtaggtatggaagatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagactcgaggaattcggtacccctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcacacacccgctctacgatatgagcctcctgaagaatcgattcctcaggccaggtgatgactccagccacgacctcatgctgctccgcctgtcagagcctgccgagctcacggatgctatgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaactccctattgtagtaaacttggaaccttggaaatgaccaggccaagactcaggcctccccagttctactgacctttgtccttaggtgtgaggtccagggttgctaggaaaagaaatcagcagacacaggtgtagaccagagtgtgggttcgaaatcgataagctctgctagcgcggggatccgcgttgcggccgcaaaaagtcgacgggcgacgcgtaaaaagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggtttttt

SEQ ID NO: 63 - DNA, 474 bp, Homo sapiens
  CDS (1)...(471)
tactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagactag

SEQ ID NO: 64 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 65 - DNA, 474 bp, Homo sapiens
  CDS (1)...(471)
tactttggcaagcttgaatctaaattatcagtcataagaaatttgaataaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaagtgaatcctcctgataacatcaaggatacaaaaagtgacatcatattctttcagagaagtgtcccaggacatgataataagatgcaatttgaatcttcatcatacgaaggatactttctaacttgtgaaaaagagagagacctttttaaactcattttgaaaaaagaggatgaattgggggatagatctataatgttcactgttcaaaacgaagactag

SEQ ID NO: 66 - protein, 157 aa, Homo sapiens
YFGKLESKLSVIRNLNNQVLFIDQGNRPLFEDMTDSDCRDNAPRTIFIISMYKDSQPRGMAVTISVKCEKISTLSCENKIISFKEVNPPDNIKDTKSDIIFFQRSVPGHDNKMQFESSSYEGYFLTCEKERDLFKLILKKEDELGDRSIMFTVQNED

SEQ ID NO: 67 - DNA, 471 bp, Homo sapiens
  CDS (1)...(471)
tactttggcaagcttgaatctaaattatcagtcataagaaatttgaatgaccaagttctcttcattgaccaaggaaatcggcctctattagaagatatgactgattctgactgtagagataatgcaccccggaccatatttattataagaatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaagcgaagac

SEQ ID NO: 68 - protein, 157 aa, Homo sapiens
YFGKLESKLSVIRNLNDQVLFIDQGNRPLLEDMTDSDCRDNAPRTIFIIRMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQSED

SEQ ID NO: 69 - DNA, 471 bp, Homo sapiens
  CDS (1)...(471)
tactttggcaagcttgcctctaaattatcagtcataagaaatttgaatgaccaagttctcttcattgaccaaggaaatcggcctctatttgaagatatgactgattctgactgtagagataatgcaccccggaccatatttattataagtatgtatgccgatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagac

SEQ ID NO: 70 - protein, 157 aa, Homo sapiens
YFGKLASKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 71 - DNA, 474 bp, Macaca mulatta
  CDS (1)...(474)
tactttggcaagcttgaatctaaattatcaatcataagaaatttgaatgaccaagttctcttcattgaccaaggaaatcggcccctatttgaagatatgactgattctgactgtagagataatgcaccccggaccatatttattataaatatgtataaagatagccagcctagaggtatggctgtagccatctctgtgaaatgtgagaaaatttcaactctctcctgtgagaacagaattatttcctttaaggaaatgaatcctcctgataacatcaaggatacgaaaagtgacatcatattctttcagagaagtgtcccaggacatgataataagatgcaatttgaatcttcatcatacgaaggatactttctagcttgtgaaaaagagagagacctttataaactcattttgaaaaaaaaggatgaattgggggatagatctataatgttcactgttcaaaacgaagactag

SEQ ID NO: 72 - protein, 157 aa, Macaca mulatta
YFGKLESKLSIIRNLNDQVLFIDQGNRPLFEDMTDSDCRDNAPRTIFIINMYKDSQPRGMAVAISVKCEKISTLSCENRIISFKEMNPPDNIKDTKSDIIFFQRSVPGHDNKMQFESSSYEGYFLACEKERDLYKLILKKKDELGDRSIMFTVQNED

SEQ ID NO: 73 - DNA, 60 bp, Homo sapiens
  CDS (1)...(60)
atggcctggaccgttctcctcctcggcctcctctctcactgcacaggctctgtgacctcc

SEQ ID NO: 74 - protein, 20 aa, Homo sapiens
MAWTVLLLGLLSHCTGSVTS

SEQ ID NO: 75 - DNA, 471 bp, Homo sapiens
  CDS (1)...(471)
tattttggcaagcttgaatctaaattatcaatcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtagctatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagac

SEQ ID NO: 76 - protein, 157 aa, Homo sapiens
YFGKLESKLSIIRNLNDQVLFIDQGNRPLFEDMTDSDCRDNAPRTIFIISMYKDSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 77 - DNA, 471 bp, Homo sapiens
  CDS (1)...(471)
tattttggcaagcttgaatctaagttaacagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtggccatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagac

SEQ ID NO: 78 - protein, 157 aa, Homo sapiens
YFGKLESKLTVIRNLNDQVLFIDQGNRPLFEDMTDSDCRDNAPRTIFIISMYKDSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 79 - DNA, 471 bp, Homo sapiens
  CDS (1)...(471)
tattttggcaagcttgaatctaagttaacagtcataagaaatttgaataaccaggttctcttcattgaccaaggaaatcggcctctatttgaagatatgactgattctgactgtagagataatgcaccccggaccatatttattataagtatgtataaagatagccagcctagaggtatggctgtggccatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagac

SEQ ID NO: 80 - protein, 157 aa, Homo sapiens
YFGKLESKLTVIRNLNNQVLFIDQGNRPLFEDMTDSDCRDNAPRTIFIISMYKDSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 81 - DNA, 5302 bp, Artificial Sequence
  rep_origin (2693)...(3592): ori
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcaggaattcgatgccgccaccatggcctggaccgttctcctcctcggcctcctctctcactgcacaggctctgtgacctcctattttggcaagcttgaatctaaattatcaatcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtagctatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagactaggacctgcagtcaccgtcgtcgacacgtgtgatcagatatcgcggccgctctagaccaggccctggatccagatctacttctggctaataaaagatcagagctctagagatctgtgtgttggttttttgtgtggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcgggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat